SEQUENCE LISTING (43 sequences)

SEQ ID NO: 1 - protein, 11 aa, Artificial Sequence
  LCDR1 Ab1 humanised anti-Clec9A antibody
QSLLHSDGNTY

SEQ ID NO: 2 - protein, 3 aa, Artificial Sequence
  LCDR2 Ab1 humanised anti-Clec9A antibody
RIS

SEQ ID NO: 3 - protein, 9 aa, Artificial Sequence
  LCDR3 Ab1 humanised anti-Clec9A antibody
LQSSHFPPT

SEQ ID NO: 4 - protein, 8 aa, Artificial Sequence
  HCDR1 Ab1 humanised anti-Clec9A antibody
GFTFNNYW

SEQ ID NO: 5 - protein, 8 aa, Artificial Sequence
  HCDR2 Ab1 humanised anti-Clec9A antibody
ITTAAGGT

SEQ ID NO: 6 - protein, 10 aa, Artificial Sequence
  HCDR3 Ab1 humanised anti-Clec9A antibody
TRVGRDIWDY

SEQ ID NO: 7 - protein, 113 aa, Artificial Sequence
  VL Ab1 humanised anti-Clec9A antibody
DIVMTQTPLSLSVTPGQPASISCKSSQSLLHSDGNTYLHWYLQKPGQPPQLLIWRISNRFSGVPDRFSGSGSGTDFTLKISRVEAEDVGVYYCLQSSHFPPTFGGGTKVEIKR

SEQ ID NO: 8 - protein, 117 aa, Artificial Sequence
  VH Ab1 humanised anti-Clec9A antibody
EVQLLESGGGLVQPGGSLRLSCAASGFTFNNYWLAWVRQAPGKGLEWVASITTAAGGTYYPDSVKGRFTISRDNSKNTLYLQMNSLRAEDTAVYYCTRVGRDIWDYWGQGTLVTVSS

SEQ ID NO: 9 - DNA, 33 bp, Artificial Sequence
  LCDR1 Ab1 humanised anti-Clec9A antibody
cagagcctgctgcacagcgacggcaatacctac

SEQ ID NO: 10 - DNA, 9 bp, Artificial Sequence
  LCDR2 Ab1 humanised anti-Clec9A antibody
cggatcagc

SEQ ID NO: 11 - DNA, 27 bp, Artificial Sequence
  LCDR3 Ab1 humanised anti-Clec9A antibody
ctgcagagcagccactttcctccaacc

SEQ ID NO: 12 - DNA, 24 bp, Artificial Sequence
  HCDR1 Ab1 humanised anti-Clec9A antibody
ggcttcaccttcaacaattattgg

SEQ ID NO: 13 - DNA, 24 bp, Artificial Sequence
  HCDR2 Ab1 humanised anti-Clec9A antibody
attaccaccgctgccggcggaacc

SEQ ID NO: 14 - DNA, 30 bp, Artificial Sequence
  HCDR3 Ab1 humanised anti-Clec9A antibody
accagagtgggcagagacatctgggattac

SEQ ID NO: 15 - DNA, 339 bp, Artificial Sequence
  VL Ab1 humanised anti-Clec9A antibody
gacatcgtgatgacccaaacacctctgagcctgagcgtgacacctggacagcctgccagcatcagctgtaaaagcagccagagcctgctgcacagcgacggcaatacctacctgcactggtatctgcagaagcccggacagcctcctcagctgctgatttggcggatcagcaacagattcagcggcgtgcccgatagattttctggctctggcagcggcaccgacttcaccctgaagattagcagagtggaagccgaggacgtgggcgtgtactactgtctgcagagcagccactttcctccaacctttggcggaggcaccaaggtggaaatcaagaga

SEQ ID NO: 16 - DNA, 351 bp, Artificial Sequence
  VH Ab1 humanised anti-Clec9A antibody
gaggtgcagctgcttgaatctggcggaggacttgttcagcctggcggctctctgagactgtcttgtgccgccagcggcttcaccttcaacaattattggctggcctgggtccgacaggcccctggaaaaggacttgagtgggtcgcctctattaccaccgctgccggcggaacctactatcccgattctgtgaagggcagattcaccatcagccgggacaacagcaagaacaccctgtacctgcagatgaacagcctgagagccgaggacaccgccgtgtactactgtaccagagtgggcagagacatctgggattactggggccagggcaccctggttacagtgtctagc

SEQ ID NO: 17 - protein, 26 aa, Artificial Sequence
  LFR1 Ab1 humanised anti-Clec9A antibody
DIVMTQTPLSLSVTPGQPASISCKSS

SEQ ID NO: 18 - protein, 17 aa, Artificial Sequence
  LFR2 Ab1 humanised anti-Clec9A antibody
LHWYLQKPGQPPQLLIW

SEQ ID NO: 19 - protein, 36 aa, Artificial Sequence
  LFR3 Ab1 humanised anti-Clec9A antibody
NRFSGVPDRFSGSGSGTDFTLKISRVEAEDVGVYYC

SEQ ID NO: 20 - protein, 11 aa, Artificial Sequence
  LFR4 Ab1 humanised anti-Clec9A antibody
FGGGTKVEIKR

SEQ ID NO: 21 - protein, 25 aa, Artificial Sequence
  HFR1 Ab1 humanised anti-Clec9A antibody
EVQLLESGGGLVQPGGSLRLSCAAS

SEQ ID NO: 22 - protein, 17 aa, Artificial Sequence
  HFR2 Ab1 humanised anti-Clec9A antibody
LAWVRQAPGKGLEWVAS

SEQ ID NO: 23 - protein, 38 aa, Artificial Sequence
  HFR3 Ab1 humanised anti-Clec9A antibody
YYPDSVKGRFTISRDNSKNTLYLQMNSLRAEDTAVYYC

SEQ ID NO: 24 - protein, 11 aa, Artificial Sequence
  HFR4 Ab1 humanised anti-Clec9A antibody
WGQGTLVTVSS

SEQ ID NO: 25 - DNA, 78 bp, Artificial Sequence
  LFR1 Ab1 humanised anti-Clec9A antibody
gacatcgtgatgacccaaacacctctgagcctgagcgtgacacctggacagcctgccagcatcagctgtaaaagcagc

SEQ ID NO: 26 - DNA, 51 bp, Artificial Sequence
  LFR2 Ab1 humanised anti-Clec9A antibody
ctgcactggtatctgcagaagcccggacagcctcctcagctgctgatttgg

SEQ ID NO: 27 - DNA, 108 bp, Artificial Sequence
  LFR3 Ab1 humanised anti-Clec9A antibody
aacagattcagcggcgtgcccgatagattttctggctctggcagcggcaccgacttcaccctgaagattagcagagtggaagccgaggacgtgggcgtgtactactgt

SEQ ID NO: 28 - DNA, 33 bp, Artificial Sequence
  LFR4 Ab1 humanised anti-Clec9A antibody
tttggcggaggcaccaaggtggaaatcaagaga

SEQ ID NO: 29 - DNA, 75 bp, Artificial Sequence
  HFR1 Ab1 humanised anti-Clec9A antibody
gaggtgcagctgcttgaatctggcggaggacttgttcagcctggcggctctctgagactgtcttgtgccgccagc

SEQ ID NO: 30 - DNA, 51 bp, Artificial Sequence
  HFR2 Ab1 humanised anti-Clec9A antibody
ctggcctgggtccgacaggcccctggaaaaggacttgagtgggtcgcctct

SEQ ID NO: 31 - DNA, 114 bp, Artificial Sequence
  HFR3 Ab1 humanised anti-Clec9A antibody
tactatcccgattctgtgaagggcagattcaccatcagccgggacaacagcaagaacaccctgtacctgcagatgaacagcctgagagccgaggacaccgccgtgtactactgt

SEQ ID NO: 32 - DNA, 33 bp, Artificial Sequence
  HFR4 Ab1 humanised anti-Clec9A antibody
tggggccagggcaccctggttacagtgtctagc

SEQ ID NO: 33 - protein, 463 aa, Artificial Sequence
  Heavy chain Ab1 humanised anti-Clec9A antibody
MDIRLSLVFLVLFIKGVQCEVQLLESGGGLVQPGGSLRLSCAASGFTFNNYWLAWVRQAPGKGLEWVASITTAAGGTYYPDSVKGRFTISRDNSKNTLYLQMNSLRAEDTAVYYCTRVGRDIWDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 238 aa, Artificial Sequence
  Light chain Ab1 humanised anti-Clec9A antibody
MKWPVRLLVLFFWIPVSRGDIVMTQTPLSLSVTPGQPASISCKSSQSLLHSDGNTYLHWYLQKPGQPPQLLIWRISNRFSGVPDRFSGSGSGTDFTLKISRVEAEDVGVYYCLQSSHFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 27 aa, Homo sapiens
RWLWQDGSSPSPGLLPAERSQSANQVC

SEQ ID NO: 36 - protein, 241 aa, Homo sapiens
MHEEEIYTSLQWDSPAPDTYQKCLSSNKCSGACCLVMVISCVFCMGLLTASIFLGVKLLQVSTIAMQQQEKLIQQERALLNFTEWKRSCALQMKYCQAFMQNSLSSAHNSSPCPNNWIQNRESCYYVSEIWSIWHTSQENCLKEGSTLLQIESKEEMDFITGSLRKIKGSYDYWVGLSQDGHSGRWLWQDGSSPSPGLLPAERSQSANQVCGYVKSNSLLSSNCSTWKYFICEKYALRSSV

SEQ ID NO: 37 - protein, 113 aa, Artificial Sequence
  VL rat anti-Clec9A antibody
EILMTQTPVSLPVSLGGQVSFSCRSSQSLLHSDGNTYLHWYLQKPGQSPQLLIWRISNRFSGVPDRFSGSGSGTDFTLKISRVEPEDLGVYYCLQSSHFPPTFGGGTNLELKR

SEQ ID NO: 38 - protein, 117 aa, Artificial Sequence
  VH human/rat chimeric anti-Clec9A antibody
EVQLVESGGGLVQPGGSLKLSCVASGFTFNNYWLAWIRQAPRKGLEWVASITTAAGGTYYPDSVKGRFTISRDNAKSTLYLQMNSLTSEDTATYYCTRVGRDIWDYWGQGVVVTVSS

SEQ ID NO: 39 - protein, 51 aa, Homo sapiens
SSGQARMFPNAPYLPSSQLECMTWNQMNLGACNKRYFKLSHLQMHSRKHTG

SEQ ID NO: 40 - protein, 247 aa, Artificial Sequence
  SARS-Cov2 RBD
KSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTG

SEQ ID NO: 41 - protein, 94 aa, Homo sapiens
LLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 42 - protein, 81 aa, Artificial Sequence
  Influenza A
MSLLTEVETPIRNEWGCRCNDSSDGGGMSLLTEVETPIRNEWGCRCNDSSDGGGMSLLTEVETPIRNEWGCRCNDSSDGGG

SEQ ID NO: 43 - protein, 113 aa, Artificial Sequence
  VL human/rat chimeric anti-Clec9A antibody
EILMTQTPVSLPVSLGGQVSFSCRSSQSLLHSDGNTYLHWYLQKPGQSPQLLIWRISNRFSGVPDRFSGSGSGTDFTLKISRVEPEDLGVYYCLQSSHFPPTFGGGTNLEIKR

(19) United States
(12) Patent Application Publication
(10) Pub. No.: US 2023/0374142 A1
(43) Pub. Date: Nov. 23, 2023

(54) CLEC9A antibodies

(71) Applicants: Monash University, Victoria (AU); The University of Queensland, Queensland (AU)

(72) Inventors: Mireille Hanna LAHOUD, Victoria (AU); Kirsteen McInnes TULLETT, Victoria (AU); Irina CAMINSCHI, Victoria (AU); Kristen Jane RADFORD, Queensland (AU); Remy ROBERT, Victoria (AU)

(21) Appl. No.: 18/029,954

(22) PCT Filed: Oct. 5, 2021
(86) PCT No.: PCT/AU2021/051160

§ 371 Date: Apr. 3, 2023

(30) Foreign Application Priority Data
Oct. 5, 2020 (AU) 2020903586

(51) Int. Cl. C07K 16/28; C12N 15/63; A61P 37/02; G01N 33/50

(52) U.S. Cl. CPC C07K 16/2851; C12N 15/63; A61P 37/02; G01N 33/5058; C07K 2317/567; C07K 2317/565; C07K 2317/622

(57) ABSTRACT

The invention relates to Clec9A, to antigen binding proteins and related fragments thereof for binding to Clec9A, to production of said antigen binding proteins and fragments and to use of said antibodies and fragments for detection and therapy of various conditions, in particular inflammation, infection and oncology.

### FIELD OF THE INVENTION

The invention relates to CLEC9A, to antigen binding proteins and related fragments thereof for binding to CLEC9A, to production of said antigen binding proteins and fragments and to use of said antibodies and fragments for detection and therapy of various conditions, in particular inflammation, infection and oncology.

### CROSS-REFERENCE TO EARLIER APPLICATION

This application claims priority from Australian provisional application no. 2020903586, the contents of which are herein incorporated by reference in their entirety.

### BACKGROUND OF THE INVENTION

Dendritic cells (DC) are bone marrow derived cells, sparsely distributed in lymphoid organs, blood and peripheral tissues, that are critical in the initiation and maintenance of an immune response. DC share common properties such as antigen (Ag) processing and the ability to activate naive T cells, initiate and maintain immune responses. However DC are heterogeneous, with multiple distinct subtypes detected in mice and humans.

DC can be broadly classified into conventional DC (cDC) and plasmacytoid DC (pDC). The pDC are able to secrete high levels of IFNα and play an important role in anti-viral responses. The cDC may be divided into the classical “migratory” DC (such as Langerhans' cells), which migrate to the lymph nodes (LN) from peripheral tissues via the lymph and the “lymphoid tissue resident” DC (found in spleen, thymus and LN), which do not migrate in this way but which arise from blood-borne precursors.

Mouse and human DC can be further subdivided into different subtypes. These DC subtypes share many functions, especially the uptake, processing and presentation of antigen (Ag) to activate naive T cells.

Importantly, DC also exhibit subset-specific roles. Different DC subtypes express different patterns of pattern recognition receptors (PRR) including Toll-like receptors (TLR) and consequently vary in their capacity to respond to different infections. As an example, the mouse and human cDC1 subsets are particularly efficient at taking up exogenous Ag, such as from dead or infected cells, and cross-presentation of these Ag on MHC class I molecules. This allows these DC to be major presenters of viral Ag to CD8+ T cells.

Molecules on the surface of DC are important in the recognition, communication and activation functions of DC. The molecules that differ between DC subtypes are of interest, since they may underpin the functional differences observed between these subtypes. Furthermore, surface molecules differing between the DC subtypes are of special interest, since they may serve as beacons for selective delivery to the DC of Ag or therapeutic agents in order to manipulate immune responses.

CLEC9A is a group V C-type lectin-like receptor (CTR) that is expressed on dendritic cells. CLEC9A can function as an endocytic receptor on a small subset of dendritic cells specialized for the uptake and processing of material from dead cells. CLEC9A recognizes filamentous form of actin, that can be associated with cytoskeletal actin-binding proteins, can be exposed when cell membranes are damaged, and can mediate the cross-presentation of dead-cell associated antigens.

Antibodies to CLEC9A have been previously described in WO2009026660, however those antibodies were derived from rats and therefore unsuitable for human use.

There exists a need for antibodies that bind to CLEC9A that can be used in humans.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides an antigen binding protein that binds to or specifically binds to CLEC9A, wherein the antigen binding protein comprises one or more sequences at least about 80%, at least 85%, at least 90%, at least 92%, at least 95%, at least 97%, at least 99%, or 100% identical to a sequence selected from the group consisting of SEQ ID NO: 17 to 24.

The invention also provides an antigen binding protein that binds to or specifically binds to CLECA, wherein the antigen binding protein comprises:
(a) a framework region (FR) 1 comprising a sequence at least about 58%, at least about 60%, at least 65%, at least 70%, at least 75%, at least 80%, at least about 85%, at least 90%, at least 95% identical to a sequence set forth in SEQ ID NO: 17, a FR2 comprising a sequence at least about 95% identical to a sequence set forth in SEQ ID NO: 18, a FR3 comprising a sequence at least about 95% identical to a sequence set forth in SEQ ID NO: 19, and a FR4 comprising a sequence at least about 73%, at least about 75%, at least 80%, at least 85%, at least 90%, at least 95% identical to a sequence set forth in SEQ ID NO: 20;
(b) a FR1 comprising a sequence at least about 88%, at least 90%, at least 95% identical to a sequence set forth in SEQ ID NO: 21, a FR2 comprising a sequence at least about 88%, at least 90%, at least 95% identical to a sequence set forth in SEQ ID NO: 22, a FR3 comprising a sequence at least about 87%, at least 90%, at least 95% identical to a sequence set forth in SEQ ID NO: 23, and a FR4 comprising a sequence at least about 82%, at least 85%, at least 90%, at least 95% identical to a sequence set forth in SEQ ID NO: 24; or
(c) each of (a) and (b).

The invention also provides an antigen binding protein that binds to or specifically binds to CLEC9A, wherein the antigen binding protein comprises:
(a) a framework region (FR) 1 comprising an amino acid sequence with 1, 2, 3, 4, 5, 6, 7, 8, 9 or 10 sequence differences compared to the amino acid set forth in SEQ ID NO: 17, a FR2 comprising an amino acid sequence with 0 amino acid differences compared to the amino acid sequence set forth in SEQ ID NO: 18, a FR3 comprising an amino acid sequence with 1 sequence difference compared to the amino acid sequence set forth in SEQ ID NO: 19, and a FR4 comprising an amino acid sequence with 1 or 2 sequence differences compared to the amino acid sequence set forth in SEQ ID NO: 20;
(b) a FR1 comprising an amino acid sequence with 1 or 2 sequence differences compared to the amino acid set forth in SEQ ID NO: 21, a FR2 comprising an amino acid sequence with 1 amino acid difference compared to the amino acid sequence set forth in SEQ ID NO: 22, a FR3 comprising an amino acid sequence with 1, 2, 3 or 4 sequence differences compared to the amino acid sequence set forth in SEQ ID NO: 23, and a FR4 comprising an amino acid sequence 1 sequence difference compared to the amino acid sequence set forth in SEQ ID NO: 24; or
(c) each of (a) and (b).

In any aspect, sequence differences may be conservative or non-conservative.

The present invention also provides an antigen binding protein that binds to or specifically binds to CLEC9A and wherein the antigen binding protein competitively inhibits binding of antibody comprising a VH comprising a sequence set forth in SEQ ID NO: 38 and a VL comprising a sequence set forth in SEQ ID NO: 37 or 43 to CLEC9A, wherein the antigen binding protein includes at least one framework region comprising a sequence at least about 60%, at least 65%, at least 70%, at least 80%, at least 85%, at least 90%, at least 92%, at least 95%, at least 97%, at least 99%, or 100% identical to 1, 2, 3, 4, 5, 6, 7 or 8 sequences selected from the group consisting of SEQ ID NO: 17-24.

The present invention also provides an antigen binding protein that binds to or specifically binds to CLEC9A and wherein the antigen binding protein competitively inhibits binding of antibody comprising a VH comprising a sequence set forth in SEQ ID NO: 8 and a VL comprising a sequence set forth in SEQ ID NO: 7 to CLEC9A, wherein the antigen binding protein includes at least one framework region comprising a sequence at least about 60%, at least 65%, at least 70%, at least 80%, at least 85%, at least 90%, at least 92%, at least 95%, at least 97%, at least 99%, or 100% identical to 1, 2, 3, 4, 5, 6, 7 or 8 sequences selected from the group consisting of SEQ ID NO: 17-24.

In any embodiment, the antigen binding protein competitively inhibits binding of an antibody to an epitope sequence set forth in SEQ ID NO: 35.

Preferably, the antigen binding protein binds to, or specifically binds to mammalian CLEC9A. Even more preferably, the antigen binding protein binds to, or specifically binds to human CLEC9A.

In any aspect, the antigen binding protein comprises complementarity determining regions (CDR):
CDRL1 has a sequence of: QSLLHSDGNTY (SEQ ID NO: 1), or a sequence with 1, 2, 3 or 4 sequence differences compared to the amino acid sequence of SEQ ID NO: 1,
CDRL2 has a sequence of: RIS (SEQ ID NO: 2), or a sequence with 1 or 2 sequence differences compared to the amino acid sequence of SEQ ID NO: 2, and
CDRL3 has a sequence of: LQSSHFPPT (SEQ ID NO: 3), or a sequence with 1, 2, 3 or 4 sequence differences compared to the amino acid sequence of SEQ ID NO: 3,
   and/or
CDRH1 has a sequence of: GFTFNNYW (SEQ ID NO: 4), or a sequence with 1, 2, 3 or 4 sequence differences compared to the amino acid sequence of SEQ ID NO: 4,
CDRH2 has a sequence of: ITTAAGGT (SEQ ID NO: 5), or a sequence with 1, 2, 3 or 4 sequence differences compared to the amino acid sequence of SEQ ID NO: 5, and
CDRH3 has a sequence of: or TRVGRDIWDY (SEQ ID NO: 6), or a sequence with 1, 2, 3 or 4 sequence differences compared to the amino acid sequence of SEQ ID NO: 6.

In any aspect, the antigen binding protein comprises CDRL1, CDRL2 and CDRL3 from SEQ ID NO: 7 and/or CDRH1, CDHRH2 and CDRH3 from SEQ ID NO:8.

In another aspect, the present invention provides an antigen binding protein that binds to or specifically binds to CLEC9A, the antigen binding protein comprises, consists essentially of or consists of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO:4, a CDR2 comprising a sequence at least about 80%, at least 85%, at least 90%, at least 92%, at least 95%, at least 97%, at least 99% identical to a sequence set forth in SEQ ID NO:5; a CDR3 comprising a sequence at least about 80%, at least 85%, at least 90%, at least 92%, at least 95%, at least 97%, at least 99% identical to a sequence set forth in SEQ ID NO: 6, and framework regions comprising 1, 2, 3 or 4 sequences at least about 60%, at least 65%, at least 70%, at least 80%, at least 85%, at least 90%, at least 92%, at least 95%, at least 97%, at least 99% identical to a sequence set forth in SEQ ID NO: 21 to 24; and/or
(ii) a VL comprising a CDR1 comprising a sequence at least about 80%, at least 85%, at least 90%, at least 92%, at least 95%, at least 97%, at least 99% identical to a sequence set forth in SEQ ID NO: 1, a CDR2 comprising a sequence at least about 80%, at least 85%, at least 90%, at least 92%, at least 95%, at least 97%, at least 99% identical to a sequence set forth in SEQ ID NO: 2, a CDR3 comprising a sequence at least about 80%, at least 85%, at least 90%, at least 92%, at least 95%, at least 97%, at least 99% identical to a sequence set forth in SEQ ID NO: 3, and framework regions comprising 1, 2, 3 or 4 sequences at least about 60%, at least 65%, at least 70%, at least 80%, at least 85%, at least 90%, at least 92%, at least 95%, at least 97%, at least 99% identical to a sequence set forth in SEQ ID NO: 17 to 20.

In another aspect, the present invention provides an antigen binding protein that binds to or specifically binds to CLEC9A, the antigen binding protein comprises, consists essentially of or consists of:
(i) a VH comprising a complementarity determining region (CDR) 1 comprising a sequence set forth in SEQ ID NO:4, a CDR2 comprising a sequence set forth in SEQ ID NO:5; a CDR3 comprising a sequence set forth in SEQ ID NO: 6, and framework regions comprising 1, 2, 3 or 4 sequences set forth in SEQ ID NO: 21 to 24; and/or
(ii) a VL comprising a CDR1 comprising a sequence forth in SEQ ID NO: 1, a CDR2 comprising a sequence set forth in SEQ ID NO: 2, a CDR3 comprising a sequence set forth in SEQ ID NO: 3, and framework regions comprising 1, 2, 3 or 4 sequences set forth in SEQ ID NO: 17 to 20.

In another aspect, the present invention provides an antigen binding protein that binds to or specifically binds to CLEC9A, the antigen binding protein comprises, consists essentially of or consists of:
(i) a VH comprising a sequence at least about 89%, at least 90%, at least 91%, at least 92%, at least 93%, at least 94%, at least 95%, at least 96%, at least 97%, at least 98% or at least 99% identical to a sequence set forth in SEQ ID NO: 8; and/or
(ii) a VL comprising a sequence at least about 85%, at least 86%, at least 87%, at least 88%, at least 89%, at least 90%, at least 91%, at least 92%, at least 93%, at least 94%, at least 95%,at least 96%, at least 97%, at least 98% or at least 99% identical to a sequence set forth in SEQ ID NO: 7.

In another aspect, the present invention provides an antigen binding protein that binds to or specifically binds to CLEC9A, the antigen binding protein comprises, consists essentially of or consists of:
(i) a VH comprising a sequence set forth in SEQ ID NO: 8; and/or
(ii) a VL comprising a sequence set forth in SEQ ID NO: 7.

In another aspect, the present invention provides an antigen binding protein that binds to or specifically binds to CLEC9A, the antigen binding protein comprises, consists essentially of or consists of:
(i) a heavy chain comprising a sequence at least at least about 89%, at least 90%, at least 91%, at least 92%, at least 93%, at least 94%, at least 95%, at least 96%, at least 97%, at least 98% or at least 99% identical to a sequence set forth in SEQ ID NO: 33, or SEQ ID NO: 33 without a leader sequence, preferably wherein the leader sequence is as shown in Table 1; and/or
(ii) a light chain comprising a sequence at least about 85%, at least 86%, at least 87%, at least 88%, at least 89%, at least 90%, at least 91%, at least 92%, at least 93%, at least 94%, at least 95%,at least 96%, at least 97%, at least 98% or at least 99% identical to a sequence set forth in SEQ ID NO: 34, or SEQ ID NO: 34 without a leader sequence, preferably wherein the leader sequence is as shown in Table 1.

In another aspect, the present invention provides an antigen binding protein that binds to or specifically binds to CLEC9A, the antigen binding protein comprises, consists essentially of or consists of:
(i) a VH comprising a sequence set forth in SEQ ID NO: 33, or SEQ ID NO: 33 without a leader sequence, preferably wherein the leader sequence is as shown in Table 1; and/or
(ii) a VL comprising a sequence set forth in SEQ ID NO: 34, or SEQ ID NO: 34 without a leader sequence, preferably wherein the leader sequence is as shown in Table 1.

In another aspect, the present invention provides an antigen binding protein that binds to or specifically binds to CLEC9A, the antigen binding protein comprising:

FR1-CDR1-FR2-CDR2-FR3-CDR3-FR4-linker-FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein:
FR1, FR2, FR3 and FR4 has a sequence at least about least about 60%, at least 65%, at least 70%, at least 80%, at least 85%, at least 90%, at least 92%, at least 95%, at least 97%, at least 99% identical or 100% identical to a sequence set forth in SEQ ID NO: 17 to 20,
FR1a, FR2a, FR3a and FR4a has a sequence least about 60%, at least 65%, at least 70%, at least 80%, at least 85%, at least 90%, at least 92%, at least 95%, at least 97%, at least 99% identical or 100% identical to a sequence set forth in SEQ ID NO: 21 to 24,and
wherein:
CDR1 has a sequence of: QSLLHSDGNTY (SEQ ID NO: 1),
CDR2 has a sequence of: RIS (SEQ ID NO: 2),
CDR3 has a sequence of: LQSSHFPPT (SEQ ID NO; 3),
CDR1a has a sequence of: GFTFNNYW (SEQ ID NO: 4),
CDR2a has a sequence of: ITTAAGGT (SEQ ID NO: 5),
CDR3a has a sequence of: TRVGRDIWDY (SEQ ID NO: 6).

In any aspect of the present invention described above, the antigen binding protein may comprise the framework regions and complementarity determining regions in the following arrangement: FR1a-CDR1a-FR2a-CDR2a-FR3a-CDR3a-FR4a-linker-FR1-CDR1-FR2-CDR2-FR3-CDR3-FR4.

As defined herein, the linker may be a chemical, one or more amino acids (including a polypeptide), or a disulphide bond formed between two cysteine residues.

In one aspect, the present invention provides an antigen binding protein that binds to, or specifically binds to CLEC9A, the antigen binding protein comprising, consisting essentially of or consisting of an amino acids sequence of (in order of N to C terminus or C to N terminus) SEQ ID NO: 7 and 8.

In one aspect, the present invention provides an antigen binding protein that binds to, or specifically binds to CLEC9A, the antigen binding protein comprising, consisting essentially of or consisting of an amino acids sequence of (in order of N to C terminus or C to N terminus) SEQ ID NO: 33 and 34 (or SEQ ID NO: 33 and 34 without a leader sequences).

In any aspect, the antigen binding protein may be in the form of:
(i) a single chain Fv fragment (scFv);
(ii) a dimeric scFv (di-scFv);
(iii) one of (i) or (ii) linked to a constant region of an antibody, Fc or a heavy chain constant domain (CH)2 and/or CH3,or
(iv) one of (i) or (ii) linked to a protein that binds to an immune effector cell.

In any aspect, the antigen binding protein may be in the form of:
(i) a diabody;
(ii) a triabody;
(iii) a tetrabody;
(iv) a bi-specific antibody;
(v) a Fab;
(vi) a F(ab′)2;
(vii) a Fv;
(viii) one of (i) to (vii) linked to a constant region of an antibody, Fc or a heavy chain constant domain (CH)2 and/or CH3,
(viii) one of (i) to (vii) linked to a protein that binds to an immune effector cell.

The present invention also provides a CLEC9A antibody comprising a light chain variable region and a heavy chain variable region,
wherein said light chain variable region comprises:
a CDR L1 as set forth in SEQ ID NO:1,a CDR L2 as set forth in SEQ ID NO:2 and a CDR L3 as set forth in SEQ ID NO:3; and
a FR L1 as set forth in SEQ ID NO:17,FR L2 as set forth in SEQ ID NO:18, a FR L3 as set forth in SEQ ID NO:19 and a FR L4 as set forth in SEQ ID NO:20, and
wherein said heavy chain variable region comprises:
a CDR H1 as set forth in SEQ ID NO:4,a CDR H2 as set forth in SEQ ID NO:5, and a CDR H3 as set forth in SEQ ID NO:6, and
a FR H1 as set forth in SEQ ID NO:21, FR H2 as set forth in SEQ ID NO:22, a FR H3 as set forth in SEQ ID NO:23 and a FR H4 as set forth in SEQ ID NO:24.

The foregoing proteins can also be referred to as antigen binding domains of antibodies. Typically, the protein is an antibody, for example, a monoclonal antibody.

In any aspect or embodiment, the antibody is a naked antibody. Specifically, the antibody is in a non-conjugated form and is not adapted to form a conjugate.

In one example, the complementarity determining region sequences (CDRs) are defined according to the IMGT numbering system.

Reference herein to a protein or antibody that “binds to” CLEC9A provides literal support for a protein or antibody that “binds specifically to” or “specifically binds to” CLEC9A.

In another aspect, the present invention also provides antigen binding domains or antigen binding fragments of the foregoing antigen binding proteins or antibodies.

In another aspect, the invention provides a fusion protein comprising an antigen binding protein as described herein. Preferably, the fusion protein further comprises a molecule, such as an antigen. In one embodiment, the antigen is fused to the N or C terminus of the VH of the antigen binding protein. In another embodiment, the antigen is fused to the N or C terminus of the VL of the antigen binding protein. In another embodiment, the antigen is fused to the N or C terminus of a constant region of the antigen binding protein. In any aspect, the antigen binding protein and other portion of the fusion protein (e.g. antigen) is separated by a linker. The linker may be any linker described herein, including a linker comprising or consisting of amino acids alanine, or glycine and serine. Exemplary linkers include AAA, AAAA and (GS)1-4.

In another aspect, the invention provides a conjugate in the form of an antigen binding protein or fusion protein as described herein conjugated to a label or a conjugated to a therapeutic agent. Examples of such agents include, but are not limited to, an antigen, a cytotoxic agent, a drug and/or pharmacological agent. In one embodiment, the antigen binding protein or fusion protein may be conjugated to a nanoparticle or emulsion for delivery of a therapeutic.

In any aspect, the antigen can be any molecule that induces an immune response in an animal. Examples include, but are not limited to, a cancer antigen, a self-antigen, an allergen, and/or an antigen from a pathogenic and/or infectious organism. Exemplary antigens as described herein.

The invention provides an antibody for binding to an antigen binding protein, fusion protein, or conjugate as described herein.

The invention provides a nucleic acid encoding an antigen binding protein, fusion protein or conjugate as described herein. Preferably, the nucleic acid has a nucleotide sequence that encodes any one or more of the amino acid sequences corresponding to SEQ ID NO: 1 to 6 and one or more sequences corresponding to SEQ ID NO: 17 to 24. Preferably, the nucleic acid as a nucleotide sequence that encodes the amino acid sequence corresponding to SEQ ID NO: 7 and/or SEQ ID NO: 8. Preferably, the nucleic acid as a nucleotide sequence that encodes the amino acid sequence corresponding to SEQ ID NO: 33 and/or SEQ ID NO: 34. In one embodiment, the nucleic acid comprises the nucleotide sequence of the VH framework regions of the antigen binding protein as shown in SEQ ID NO: 16. In another embodiment, the nucleic acid comprises the nucleotide sequence of the VL framework regions of the antigen binding protein as shown in SEQ ID NO: 15. In any embodiment, the nucleic acid comprises the nucleotide sequences shown in SEQ ID Nos: 25, 26, 27 and/or 28. In another embodiment, the nucleic acid comprises the nucleotides sequence shown in SEQ ID Nos: 29, 30, 31 and/or 32. In a further embodiment, the nucleic acid comprises the nucleotide sequences shown in SEQ ID Nos: 25 to 32. In any aspect or embodiment, the nucleic acid further comprises the nucleotide sequences of SEQ ID Nos: 9 to 11, 12 to 14, or 9 to 14.

In another aspect, the nucleic acid comprises the nucleotide sequence as shown in SEQ ID NO: 16 and/or 15.

In one example, such a nucleic acid is included in an expression construct in which the nucleic acid is operably linked to a promoter. Such an expression construct can be in a vector, e.g., a plasmid.

In any aspect, the nucleic acid is DNA or RNA. In one embodiment, the nucleic acid is mRNA.

In examples of the invention directed to single polypeptide chain antigen binding protein, the expression construct may comprise a promoter linked to a nucleic acid encoding that polypeptide chain.

In examples directed to multiple polypeptide chains that form an antigen binding protein, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide,
wherein the first polypeptide comprises a VH and the second polypeptide comprises a VL, or vice versa.

The present invention also contemplates separate expression constructs one of which encodes a first polypeptide comprising a VH and another of which encodes a second polypeptide comprising a VL. For example, the present invention also provides a composition comprising:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter.

The invention provides a cell comprising a vector or nucleic acid described herein. Preferably, the cell is isolated, substantially purified or recombinant. In one example, the cell comprises the expression construct of the invention or:
(i) a first expression construct comprising a nucleic acid encoding a polypeptide comprising a VH operably linked to a promoter; and
(ii) a second expression construct comprising a nucleic acid encoding a polypeptide comprising a VL operably linked to a promoter,
wherein the first and second polypeptides associate to form an antigen binding protein of the present invention.

Examples of cells of the present invention include bacterial cells, yeast cells, insect cells or mammalian cells.

In another aspect, the invention provides a pharmaceutical composition comprising an antigen binding protein, fusion protein, or conjugate as described herein and a pharmaceutically acceptable carrier, diluent or excipient. Preferably, the pharmaceutical composition further comprises an adjuvant or DC activating agent.

In another aspect, the pharmaceutical composition does not contain any dendritic cell activating agent or adjuvant other than the antigen binding protein, fusion protein, or conjugate as described herein.

In another aspect, the invention provides a diagnostic composition comprising an antigen binding protein, fusion protein or conjugate as described herein, a diluent and optionally a label.

In another aspect, the invention provides a kit or article of manufacture comprising an antigen binding protein, fusion protein or conjugate as described herein.

An antigen binding protein described herein may comprise a human constant region, e.g., an IgG constant region, such as an IgG1, IgG2, IgG3 or IgG4 constant region or mixtures thereof. In the case of an antibody or protein comprising a VH and a VL, the VH can be linked to a heavy chain constant region and the VL can be linked to a light chain constant region.

In one example, a protein or antibody as described herein comprises a constant region of an IgG4 antibody or a stabilized constant region of an IgG4 antibody. In one example, the protein or antibody comprises an IgG4 constant region with a proline at position 241 (according to the numbering system of Kabat (Kabat et al., Sequences of Proteins of Immunological Interest Washington DC United States Department of Health and Human Services, 1987 and/or 1991)), or position 244 numbering as per SEQ ID NO: 33.

In one example, an antibody of the invention comprises a VH disclosed herein linked or fused to an IgG4 constant region or stabilized IgG4 constant region (e.g., as discussed above) and the VL is linked to or fused to a kappa light chain constant region.

The functional characteristics of an antigen binding protein of the invention will be taken to apply mutatis mutandis to an antibody of the invention.

In one example, an antigen binding protein or antibody as described herein is isolated, substantially purified and/or recombinant.

In one example, an antigen binding protein or antibody of the invention is conjugated to another compound, for example, a detectable label or a compound that extends the half-life of the protein or antibody, such as polyethylene glycol or an albumin binding protein. Other suitable compounds are described herein.

In another aspect, the present invention additionally provides methods for producing an antigen binding protein or antibody of the invention. For example, such a method involves maintaining the expression construct(s) of the invention under conditions sufficient for the antigen binding protein or antibody to be produced.

In one example, a method for producing an antigen binding protein or antibody of the invention comprises culturing the cell of the invention under conditions sufficient for the antigen binding protein or antibody to be produced and, optionally, secreted.

In one example, the method for producing an antigen binding protein or antibody of the invention additionally comprises isolating the protein or antibody and, optionally, formulating the antigen binding protein or antibody into a pharmaceutical composition.

The present invention additionally provides a composition comprising an antigen binding protein or antibody as described herein and a pharmaceutically acceptable carrier.

In another aspect, the present invention also provides a complex comprising an antigen binding protein of the invention and CLEC9A. In one example, the CLEC9A is recombinant CLEC9A.

In another aspect, the present invention provides a method of modulating an immune response in a subject, the method comprising administering to the subject an antigen binding protein, fusion protein, conjugate or pharmaceutical composition of the invention, thereby modulating an immune response in a subject.

In an embodiment, the immune response to an antigen is induced and/or enhanced. Preferably, the immune response to the antigen that is part of the fusion protein or conjugate is induced and/or enhanced.

In a particularly preferred embodiment, the immune response is modulated by enhancing a helper T cell response.

In a further preferred embodiment, the immune response is modulated by the activation of CD4+ and/or CD8+ T cells.

In another particularly preferred embodiment, the immune response is modulated by enhancing B cell antibody production. Examples of antibodies produced include, but are not necessarily limited to, IgG1, IgG2, IgG3 and/or IgG4 antibody isotypes.

In a further preferred embodiment, the immune response is modulated by generating a memory response.

In a further aspect, the present invention provides a method of modulating an immune response to an antigen in a subject, the method comprising exposing dendritic cells or precursors thereof in vitro to an antigen binding protein, fusion protein, conjugate or pharmaceutical composition of the invention, and administering said cells to the subject, thereby modulating an immune response to the antigen in the subject.

In an embodiment, the cells have been isolated from the subject. Preferably, a humoral and/or T cell mediated response is modulated.

In a further embodiment, naive CD8+ T cell activation, and/or naive CD4+ T cell activation, is modulated.

In yet another embodiment, the humoral response comprises the production of IgG1, IgG2, IgG3 and/or IgG4 antibody isotypes.

In another embodiment, the humoral response at least comprises the production of IgG1 antibody isotype.

Preferably, the dendritic cell is an animal dendritic cell or precursor of an animal dendritic cell. More preferably, the dendritic cell is a human dendritic cell. Even more preferably, the human dendritic cell is NECL-2+, HLA DR+, XCR-1+, CLEC9A+ and/or BDCA-3+.

In yet another aspect, the present invention provides a method of treating and/or preventing a disease involving dendritic cells or precursors thereof, the method comprising administering to the subject an antigen binding protein, fusion protein, conjugate or pharmaceutical composition of the invention, thereby treating and/or preventing a disease involving dendritic cells or precursors thereof.

Examples of diseases involving dendritic cells or precursors thereof include, but are not limited to, cancer, an infection, an autoimmune disease or an allergy. Examples of infectious disease include coronavirus (e.g. SARS-CoV-2), influenza, Dengue, hand-foot-mouth disease.

The antigen binding protein, fusion protein, conjugate or pharmaceutical composition of the invention may be used prophylactically or in a preventive method in the context of infectious disease. Alternatively, the antigen binding protein, fusion protein, conjugate or pharmaceutical composition of the invention may be used therapeutically or in a treatment method in the context of cancer.

In another aspect, the present invention provides for the use of an antigen binding protein, fusion protein, conjugate or pharmaceutical composition of the invention for the manufacture of a medicament for modulating an immune response in a subject.

In a further aspect, the present invention provides for the use of dendritic cells or precursors thereof exposed in vitro to an antigen binding protein, fusion protein, conjugate or pharmaceutical composition of the invention for the manufacture of a medicament for modulating an immune response to an antigen in a subject.

In yet another aspect, the present invention provides for the use of an antigen binding protein, fusion protein, conjugate or pharmaceutical composition of the invention for the manufacture of a medicament for treating and/or preventing a disease involving dendritic cells or precursors thereof in a subject.

In another aspect, the present invention provides an antigen binding protein, fusion protein, conjugate or pharmaceutical composition of the invention for use in modulating an immune response to an antigen in a subject.

In another aspect, the present invention provides an antigen binding protein, fusion protein, conjugate or pharmaceutical composition of the invention for use in treating and/or preventing a disease involving dendritic cells or precursors thereof

In a further aspect, the present invention provides a method of enriching-dendritic cells, or a subset or precursors thereof, from a sample comprising;
(i) contacting a sample comprising dendritic cells or precursors thereof with an antigen binding protein, fusion protein, conjugate or pharmaceutical composition of the invention, and
(ii) isolating cells bound to the antigen binding protein, fusion protein, conjugate of the invention.

In a further aspect, the present invention provides a method of detecting dendritic cells, or a subset or precursors thereof, in a sample comprising;
(i) contacting a sample comprising dendritic cells or precursors thereof with an antigen binding protein, fusion protein, conjugate of the invention,
(ii) detecting cells bound to the antigen binding protein, fusion protein, conjugate of the invention.

In a preferred embodiment, the dendritic cells express one or more of the following markers, CD8, CD24, NECL-2 (otherwise referred to as cell adhesion molecule 1 (CADM1), BL2, IGSF4, RA175, ST17, SYNCAM, TSLC1), CD11c (otherwise referred to as Integrin alpha X), HLADR, XCR-1 (receptor for XCL1-1, otherwise referred to as ATAC, lymphotactin-1 or SCM-1), CLEC9A and BDCA3 (also referred to as thrombomodulin, THBD, AHUS6, THPH12, THRM, TM, CD141).

Preferably, the dendritic cells are human dendritic cells that express one or more of the following markers, NECL-2, HLADR, XCR-1, CLEC9A and BDCA3.

Preferably, the precursor dendritic cells are intermediate or late precursor dendritic cells which are capable of differentiating into dendritic cells in culture and/or on transfer into irradiated recipients.

Preferably, the subject is an animal. More preferably, the subject is a mammal such as a human, dog, cat, horse, cow, or sheep. Most preferably, the subject is a human.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: A. Binding of purified humanised anti-CLEC9A Ab by ELISA. Plates were coated with soluble human CLEC9A and binding of human/rat chimaeric anti-CLEC9A Ab and humanised anti-CLEC9A Ab were detected using anti-human IgG4-biotin and streptavidin HRP. Data is representative of 6 independent experiments.

B. Binding of humanised anti-CLEC9A Ab to cell surface CLEC9A. 293F cells were transfected with expression constructs encoding human CLEC9A and binding of the humanised anti-CLEC9A Ab, at concentrations indicated, was detected using anti-human IgG4-biotin and streptavidin-PE by flow cytometry.
C. Binding of humanised anti-CLEC9A Ab to human blood DC. Human blood DC enriched from PBMC were stained with anti-CLEC9A Ab, detected with anti-human IgG4-biotin and streptavidin-PE, and with markers to distinguish cDC1 (CD141), cDC2 (CD1c) and pDC (CD123). Data is representative of 2 independent experiments.

FIG. 2: A. and B. Binding of purified humanised anti-CLEC9A Ab-Ag by ELISA. Plates were coated with soluble CLEC9A and Ab binding was detected with anti-human IgG4-biotin and streptavidin HRP for anti-CLEC9A-WT1 (A) or anti-CLEC9A-RBD (B). Data is representative of 2 independent experiments.

C. Humanised anti-CLEC9A, anti-CLEC9A-WT1, anti-CLEC9A-M2e, anti-CLEC9A-NY-ESO-1 and anti-CLEC9A-RBD were expressed, purified, and Ab binding (5μg/ml) to 293F cells transfected with human CLEC9A, or an un-transfected control detected with anti-human IgG4-biotin and streptavidin-PE by flow cytometry. Data is representative of 2 independent experiments.
D. Binding of humanised anti-CLEC9A Ab-Ag to human blood DC. Human blood DC enriched from PBMC were stained with 10 μg/ml anti-CLEC9A-Ab or anti-CLEC9A-Ab-Ag, detected with anti-human IgG4-biotin and streptavidin-PE, and with markers to distinguish cDC1 (CD141), cDC2 (CD1c) and pDC (CD123). Data representative of 3 independent experiments for anti-CLEC9A Ab, anti-CLEC9A-WT1 and anti-CLEC9A-RBD and one experiment for anti-CLEC9A-M2e and anti-CLEC9A-NY-ESO-1.

FIG. 3: Humanised mice developing a repertoire of human naïve WT1235-243-specific CD8+ T cells and human dendritic cell subsets in spleens were generated. Splenocytes were isolated and cultured with humanised anti-CLEC9A-WT1 antibodies and controls (10 μg/ml). Data shown are the mean +SD from triplicates and are representative of 2 independent experiments. T cell activation was measured by IFNγ ELISPOT after overnight culture.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present inventors have developed antigen binding proteins, for example antibodies, that bind to CLEC9A. The inventors have also developed antibodies that bind to CLEC9A, preferably human CLEC9A, but that have reduced immunogenicity in humans due to modification of framework regions. Specifically, the framework regions have been modified, or humanised, to reduce the likelihood that a human subject will raise an immune response against the antibody.

Surprisingly, the antibody with modifications to the framework regions still retains potent affinity to CLEC9A, and specificity, and does so when fused to antigenic amino acid sequences. For example, the inventors have generated fusion proteins comprising humanised CLEC9A antibodies fused to tumour antigens WT1 and NY-ESO-1 or the infectious disease antigens SARS-CoV2 RBD and influenza M2e (extracellular domain of M2 (M2e)). Therefore, the antigen binding proteins of the invention provide a means of delivering a payload, such as vaccine/tumour antigens or other cargo, to human dendritic cells both in vitro and in vivo for the induction of immune responses.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901 -917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein the term “derived from” shall be taken to indicate that a specified integer may be obtained from a particular source albeit not necessarily directly from that source.

Reference herein to a range of, e.g., residues, will be understood to be inclusive. For example, reference to “a region comprising amino acids 56 to 65” will be understood in an inclusive manner, i.e., the region comprises a sequence of amino acids as numbered 56, 57, 58, 59, 60, 61, 62, 63, 64 and 65 in a specified sequence.

### Selected Definitions

CLEC9A (also known as DNGR1, UNQ9341, CD370, DNGR-1, C-type lectin domain family 9 member A, C-type lectin domain containing 9A) is a group V C-type lectin-like receptor (CLR) that functions as an activation receptor and is expressed on dendritic cells. CLEC9A can function as an endocytic receptor on a small subset of dendritic cells specialized for the uptake and processing of material from dead cells. CLEC9A recognizes filamentous form of actin, that can be associated with actin-binding proteins, can be exposed when cell membranes are damaged, and can mediate the cross-presentation of dead-cell associated antigens.

The term “CLEC9A” as provided herein includes any of the CLEC9A protein naturally occurring forms, homologs or variants that maintain the activity of CLEC9A (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In embodiments, the CLEC9A protein is the protein as identified by SEQ ID NO: 36, homolog or functional fragment thereof.

For the purposes of nomenclature only and not a limitation, an exemplary amino acid sequence of human CLEC9A is SEQ ID NO: 36.

As used herein, the term “C-type lectin-like domain” or “CTLD” refers to a protein domain family which has been identified in a number of proteins isolated from many animal species. Initially, the CTLD domain was identified as a domain common to the so-called C-type lectins (calcium-dependent carbohydrate binding proteins) and named “Carbohydrate Recognition Domain” (“CRD”). More recently, it has become evident that this domain is shared among many eukaryotic proteins, of which several do not bind sugar moieties, and hence, the canonical domain has been named as CTLD. CTLDs have been reported to bind a wide diversity of compounds, including carbohydrates, lipids and proteins. The CTLD consists of approximately 120 amino acid residues and, characteristically, contains two or three intra-chain disulphide bridges. Although the similarity at the amino acid sequence level between CTLDs from different proteins is relatively low, the 3D-structures of a number of CTLDs have been found to be highly conserved, with the structural variability essentially confined to a so-called loop-region, often defined by up to five loops.

As used herein, the term “immune response” refers to an alteration in the reactivity of the immune system of a subject in response to an antigen and may involve antibody production, induction of cell-mediated immunity, complement activation and/or development of immunological tolerance.

As used herein, the “sample” can be any biological material suspected of comprising dendritic cells or precursors thereof. Examples include, but are not limited to, blood, for example, whole peripheral blood, cord blood, foetus blood, bone marrow, plasma, serum, urine, cultured cells, saliva or urethral swab, lymphoid tissues, for example tonsils, Peyer's patches, appendix, thymus, spleen and lymph nodes. The sample may be tested directly or may require some form of treatment prior to testing. For example, a biopsy sample may require homogenization to produce a cell suspension prior to testing. Furthermore, to the extent that the biological sample is not in liquid form (for example, it may be a solid, semi-solid or a dehydrated liquid sample), it may require the addition of a reagent, such as a buffer, to mobilize the sample. The mobilizing reagent may be mixed with the sample prior to placing the sample in contact with, for example, an antigen binding protein of the invention.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding protein” is used interchangeably with “antigen binding domain” and shall be taken to mean a region of an antibody that is capable of specifically binding to an antigen, i.e., a VH or a VL or an Fv comprising both a VH and a VL. The antigen binding domain need not be in the context of an entire antibody, e.g., it can be in isolation (e.g., a domain antibody) or in another form, e.g., as described herein, such as a scFv.

For the purposes for the present disclosure, the term “antibody” includes a protein capable of specifically binding to one or a few closely related antigens (e.g., CLEC9A) by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (˜50 to 70 kD) covalently linked and two light chains (˜23 kDa each). A light chain generally comprises a variable region (if present) and a constant domain and in mammals is either a K light chain or a λ light chain. A heavy chain generally comprises a variable region and one or two constant domain(s) linked by a hinge region to additional constant domain(s). Heavy chains of mammals are of one of the following types α, δ, ⊖, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ˜110 amino acids in length) and one or more constant domains at the C-terminus. The constant domain of the light chain (CL which is ˜110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example, the antibody heavy chain is missing a C-terminal lysine residue. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1 (or HCDR1), CDR H2 (or HCDR2) and CDR H3 (or HCDR3), respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1 (or LCDR1), CDR L2 (or LCDR2) and CDR L3 (or LCDR3), respectively, wherein CDR L1 corresponds to CDR 1 of VL, CDR L2 corresponds to CDR 2 of VL and CDR L3 corresponds to CDR 3 of VL. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plukthun J. Mol. Biol. 309: 657-670, 2001; or the IMGT system discussed in Giudicelli et al., Nucleic Acids Res. 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 are not generally involved in antigen binding.

“Framework regions” (FRs) are those variable region residues other than the CDR residues. The FRs of VH are also referred to herein as FR H1 (or HFR1), FR H2 (or HFR2), FR H3 (or HFR3) and FR H4 (or HFR4), respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of VL are referred to herein as FR L1 (or LFR1), FR L2 (or LFR2), FR L3 (or LFR3) and FR L4 (or LFR4), respectively, wherein FR L1 corresponds to FR 1 of VL, FR L2 corresponds to FR 2 of VL, FR L3 corresponds to FR 3 of VL and FR L4 corresponds to FR 4 of VL.

As used herein, the term “Fv” shall be taken to mean any protein, whether comprised of multiple polypeptides or a single polypeptide, in which a VL and a VH associate and form a complex having an antigen binding domain, i.e., capable of specifically binding to an antigen. The VH and the VL which form the antigen binding domain can be in a single polypeptide chain or in different polypeptide chains. Furthermore, an Fv of the invention (as well as any protein of the invention) may have multiple antigen binding domains which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of an antigen binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labeled “A” and the protein, will reduce the amount of labelled “A” bound to the antibody.

As used herein, the term “specifically binds” or “binds specifically” shall be taken to mean that an antigen binding protein of the invention reacts or associates more frequently, more rapidly, with greater duration and/or with greater affinity with a particular antigen or cell expressing same than it does with alternative antigens or cells. For example, an antigen binding protein binds to CLEC9A (e.g., human CLEC9A) with materially greater affinity (e.g., 1.5 fold or 2 fold or 5 fold or 10 fold or 20 fold or 40 fold or 60 fold or 80 fold to 100 fold or 150 fold or 200 fold) than it does to one or more other closely related proteins. In an example of the present invention, an antigen binding protein that “specifically binds” to CLEC9A (preferably human) with an affinity at least 1.5 fold or 2 fold or greater (e.g., 5 fold or 10 fold or 20 fold or 50 fold or 100 fold or 200 fold) than it does to another protein comprising a CTLD. Generally, but not necessarily, reference to binding means specific binding, and each term shall be understood to provide explicit support for the other term.

As used herein, the term “does not detectably bind” shall be understood to mean that an antigen binding protein, e.g., an antibody, binds to a candidate antigen at a level less than 10%, or 8% or 6% or 5% above background. The background can be the level of binding signal detected in the absence of the protein and/or in the presence of a negative control protein (e.g., an isotype control antibody) and/or the level of binding detected in the presence of a negative control antigen. The level of binding is detected using binding assays know in the field, e.g. biosensor analysis(e.g. Biacore), flow cytometry, ELISA etc in which the antigen binding protein is immobilized and contacted with an antigen, or when the antigen is expressed on the surface of the cell and binding detected using flow cytometry.

As used herein, the term “does not significantly bind” shall be understood to mean that the level of binding of an antigen binding protein of the invention to a polypeptide is not statistically significantly higher than background, e.g., the level of binding signal detected in the absence of the antigen binding protein and/or in the presence of a negative control protein (e.g., an isotype control antibody) and/or the level of binding detected in the presence of a negative control polypeptide. The level of binding is detected using binding assays know in the field, e.g. biosensor analysis(e.g. Biacore), flow cytometry, ELISA etc in which the antigen binding protein is immobilized and contacted with an antigen, or when the antigen is expressed on the surface of the cell and binding detected using flow cytometry.

As used herein, the term “epitope” (syn. “antigenic determinant”) shall be understood to mean a region of CLEC9A to which an antigen binding protein comprising an antigen binding domain of an antibody binds. Unless otherwise defined, this term is not necessarily limited to the specific residues or structure to which the antigen binding protein makes contact. For example, this term includes the region spanning amino acids contacted by the antigen binding protein and 5-10 (or more) or 2-5 or 1-3 amino acids outside of this region. In some examples, the epitope comprises a series of discontinuous amino acids that are positioned close to one another when antigen binding protein is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

As used herein, the term “condition” refers to a disruption of or interference with normal function, and is not to be limited to any specific condition, and will include diseases or disorders.

As used herein, the terms “preventing”, “prevent” or “prevention” include administering an antigen binding protein of the invention to thereby stop or hinder the development of at least one symptom of a condition. This term also encompasses treatment of a subject in remission to prevent or hinder relapse.

As used herein, the terms “treating”, “treat” or “treatment” include administering an antigen binding protein described herein to thereby reduce or eliminate at least one symptom of a specified disease or condition.

As used herein, the term “subject” shall be taken to mean any animal including humans, for example a mammal. Exemplary subjects include but are not limited to humans and non-human primates. For example, the subject is a human.

### Antibodies

In one example, an antigen binding protein or CLEC9A-binding protein as described herein according to any example is an antibody.

Methods for generating antibodies are known in the art and/or described in Harlow and Lane (editors) Antibodies: A Laboratory Manual, Cold Spring Harbor Laboratory, (1988). Generally, in such methods CLEC9A (e.g., human CLEC9A) or a region thereof (e.g., an extracellular region) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, subcutaneously, intravenously, intradermally, intraperitoneally, or by other known route.

The production of polyclonal antibodies may be monitored by sampling blood of the immunized animal at various points following immunization. One or more further immunizations may be given, if required to achieve a desired antibody titer. The process of boosting and titering is repeated until a suitable titer is achieved. When a desired level of immunogenicity is obtained, the immunized animal is bled and the serum isolated and stored, and/or the animal is used to generate monoclonal antibodies (mAbs).

Monoclonal antibodies are one exemplary form of antibody contemplated by the present invention. The term “monoclonal antibody” or “mAb” refers to a homogeneous antibody population capable of binding to the same antigen(s), for example, to the same epitope within the antigen. This term is not intended to be limited with regard to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

For example, a suitable animal is immunized with an immunogen under conditions sufficient to stimulate antibody producing cells. Rodents such as rabbits, mice and rats are exemplary animals. Mice genetically-engineered to express human antibodies, for example, which do not express murine antibodies, can also be used to generate an antibody of the present invention (e.g., as described in WO2002/066630).

Following immunization, somatic cells with the potential for producing antibodies, specifically B lymphocytes (B cells), are selected for use in the mAb generating protocol. These cells may be obtained from biopsies of spleens, tonsils or lymph nodes, or from a peripheral blood sample. The B cells from the immunized animal are then fused with cells of an immortal myeloma cell, generally derived from the same species as the animal that was immunized with the immunogen.

Hybrids are amplified by culture in a selective medium comprising an agent that blocks the de novo synthesis of nucleotides in the tissue culture media. Exemplary agents are aminopterin, methotrexate and azaserine.

The amplified hybridomas are subjected to a functional selection for antibody specificity and/or titer, such as, for example, by flow cytometry and/or immunohistochemstry and/or immunoassay (e.g. radioimmunoassay, enzyme immunoassay, cytotoxicity assay, plaque assay, dot immunoassay, and the like).

Alternatively, ABL-MYC technology (NeoClone, Madison WI 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. Nos. 6,300,064 and/or 5,885,793. For example, the present inventors have isolated fully human antibodies from a phage display library.

The antibody of the present invention may be a synthetic or recombinant antibody.

### Antibody Binding Domain Containing Proteins

### Single-Domain Antibodies

In some examples, a protein of the invention is or comprises a single-domain antibody (which is used interchangeably with the term “domain antibody” or “dAb”). A single-domain antibody is a single polypeptide chain comprising all or a portion of the heavy chain variable region of an antibody. In certain examples, a single-domain antibody is a human single-domain antibody (Domantis, Inc., Waltham, MA; see, e.g., U.S. Pat. No. 6,248,516).

### Diabodies, Triabodies, Tetrabodies

In some examples, a protein of the invention is or comprises a diabody, triabody, tetrabody or higher order protein complex such as those described in WO98/044001 and/or WO94/007921.

For example, a diabody is a protein comprising two associated polypeptide chains, each polypeptide chain comprising the structure VL-X-VH or VH-X-VL, wherein VL is an antibody light chain variable region, VH is an antibody heavy chain variable region, X is a linker comprising insufficient residues to permit the VH and VL in a single polypeptide chain to associate (or form an Fv) or is absent, and wherein the VH of one polypeptide chain binds to a VL of the other polypeptide chain to form an antigen binding domain, i.e., to form a Fv molecule capable of specifically binding to one or more antigens. The VL and VH can be the same in each polypeptide chain or the VL and VH can be different in each polypeptide chain so as to form a bispecific diabody (i.e., comprising two Fvs having different specificity).

### Single Chain Fv (scFv)

The skilled artisan will be aware that scFvs comprise VH and VL regions in a single polypeptide chain and a polypeptide linker between the VH and VL which enables the scFv to form the desired structure for antigen binding (i.e., for the VH and VL of the single polypeptide chain to associate with one another to form a Fv). For example, the linker comprises in excess of 12 amino acid residues with (Gly4Ser)3 being one of the more favored linkers for a scFv.

The present invention also contemplates a disulfide stabilized Fv (or diFv or dsFv), in which a single cysteine residue is introduced into a FR of VH and a FR of VL and the cysteine residues linked by a disulfide bond to yield a stable Fv.

Alternatively, or in addition, the present invention encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

### Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

The variable regions present in naturally occurring heavy chain antibodies are generally referred to as “VHH domains” in camelid antibodies and V-NAR in IgNAR, in order to distinguish them from the heavy chain variable regions that are present in conventional 4-chain antibodies (which are referred to as “VH domains”) and from the light chain variable regions that are present in conventional 4-chain antibodies (which are referred to as “VL domains”).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

### Other Antibodies and Proteins Comprising Antigen Binding Domains Thereof

The present invention also contemplates other antibodies and proteins comprising antigen-binding domains thereof, such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab3 (e.g., as described in EP19930302894).

### Mutations to Proteins

The present invention also provides an antigen binding protein or a nucleic acid encoding same having at least 80% identity to a sequence disclosed herein. In one example, an antigen binding protein or nucleic acid of the invention comprises sequence at least about 85% or 90% or 95% or 97% or 98% or 99% identical to a sequence disclosed herein.

Alternatively, or additionally, the antigen binding protein comprises a CDR (e.g., three CDRs) at least about 80% or 85% or 90% or 95% or 97% or 98% or 99% identical to CDR(s) of a VH or VL as described herein according to any aspect, embodiment or example.

In another example, a nucleic acid of the invention comprises a sequence at least about 80% or 85% or 90% or 95% or 97% or 98% or 99% identical to a sequence encoding an antigen binding protein having a function as described herein according to any aspect, embodiment or example. The present invention also encompasses nucleic acids encoding an antigen binding protein of the invention, which differs from a sequence exemplified herein as a result of degeneracy of the genetic code.

The % identity of a nucleic acid or polypeptide is determined by GAP (Needleman and Wunsch. Mol. Biol. 48, 443-453, 1970) analysis (GCG program) with a gap creation penalty=5, and a gap extension penalty=0.3. The query sequence is at least 50 residues in length, and the GAP analysis aligns the two sequences over a region of at least 50 residues. For example, the query sequence is at least 100 residues in length and the GAP analysis aligns the two sequences over a region of at least 100 residues. For example, the two sequences are aligned over their entire length.

The present invention also contemplates a nucleic acid that hybridizes under stringent hybridization conditions to a nucleic acid encoding an antigen binding protein described herein. A “moderate stringency” is defined herein as being a hybridization and/or washing carried out in 2 x SSC buffer, 0.1% (w/v) SDS at a temperature in the range 45*C to 65*C, or equivalent conditions. A “high stringency” is defined herein as being a hybridization and/or wash carried out in 0.1×SSC buffer, 0.1% (w/v) SDS, or lower salt concentration, and at a temperature of at least 65*C, or equivalent conditions. Reference herein to a particular level of stringency encompasses equivalent conditions using wash/hybridization solutions other than SSC known to those skilled in the art. For example, methods for calculating the temperature at which the strands of a double stranded nucleic acid will dissociate (also known as melting temperature, or Tm) are known in the art. A temperature that is similar to (e.g., within 5μ C. or within 10μ C.) or equal to the Tm of a nucleic acid is considered to be high stringency. Medium stringency is to be considered to be within 10° C. to 20° C. or 10β C. to 15° C. of the calculated Tm of the nucleic acid.

The present invention also contemplates mutant forms of an antigen binding protein of the invention comprising one or more conservative amino acid substitutions compared to a sequence set forth herein. In some examples, the antigen binding protein comprises 10 or fewer, e.g., 9 or 8 or 7 or 6 or 5 or 4 or 3 or 2 or 1 conservative amino acid substitutions. A “conservative amino acid substitution” is one in which the amino acid residue is replaced with an amino acid residue having a similar side chain and/or hydropathicity and/or hydrophilicity. As used herein, a “sequence difference” may be a conservative substitution.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophylic indices are described in, e.g., U.S. Pat. No. 4,554,101.

The present invention also contemplates non-conservative amino acid changes. For example, of particular interest are substitutions of charged amino acids with another charged amino acid and with neutral or positively charged amino acids. In some examples, the antigen binding protein comprises 10 or fewer, e.g., 9 or 8 or 7 or 6 or 5 or 4 or 3 or 2 or 1 non-conservative amino acid substitutions. As used herein, a “sequence difference” may be a non-conservative substitution.

In one example, the mutation(s) occur within a FR of an antigen binding domain of an antigen binding protein of the invention. In another example, the mutation(s) occur within a CDR of an antigen binding protein of the invention.

Exemplary methods for producing mutant forms of an antigen binding protein include:
mutagenesis of DNA (Thie et al., Methods Mol. Biol. 525: 309-322, 2009) or RNA (Kopsidas et al., Immunol. Lett. 107:163-168, 2006; Kopsidas et al. BMC Biotechnology, 7: 18, 2007; and WO1999/058661);
introducing a nucleic acid encoding the polypeptide into a mutator cell, e.g., XL-1Red, XL-mutS and XL-mutS-Kanr bacterial cells (Stratagene);
DNA shuffling, e.g., as disclosed in Stemmer, Nature 370: 389-91, 1994; and
site directed mutagenesis, e.g., as described in Dieffenbach (ed) and Dveksler (ed) (In: PCR Primer: A Laboratory Manual, Cold Spring Harbor Laboratories, NY, 1995).

Exemplary methods for determining biological activity of the mutant antigen binding proteins of the invention will be apparent to the skilled artisan and/or described herein, e.g., antigen binding. For example, methods for determining antigen binding, competitive inhibition of binding, affinity, association, dissociation and therapeutic efficacy are described herein.

### Constant Regions

The present invention encompasses antigen binding proteins and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG4 Fc region (i.e., from an IgG4 constant region), e.g., a human IgG4 Fc region. Sequences of suitable IgG4 Fc regions will be apparent to the skilled person and/or available in publicly available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S-S) bonds in the same positions (see for example WO2010/080538). As shown herein, P at position 244 (often referred to as S246P) is a mutation to stabilise the hinge numbering as per SEQ ID NO: 33.

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol ;75: 12161-12168, 2001).

In another example, the Fc region is a chimeric Fc region, e.g., comprising at least one CH2 domain from an IgG4 antibody and at least one CH3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Additional Modifications

The present invention also contemplates additional modifications to an antibody or antigen binding protein comprising an Fc region or constant region.

For example, the antibody comprises one or more amino acid substitutions that increase the half-life of the protein. For example, the antibody comprises a Fc region comprising one or more amino acid substitutions that increase the affinity of the Fc region for the neonatal Fc region (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

Alternatively, the invention also contemplates additional modifications that reduce or inhibit FcR binding, for example an E at position 251 (often referred to as L253E) numbering as per SEQ ID NO: 33.

### Antigens

The term “antigen” is further intended to encompass peptide or protein analogs of known or wild-type antigens such as those described above. The analogs may be more soluble or more stable than wild type antigen, and may also contain mutations or modifications rendering the antigen more immunologically active. Also useful in the present invention are peptides or proteins which have amino acid sequences homologous with a desired antigen's amino acid sequence, where the homologous antigen induces an immune response to the respective tumour or organism.

A “cancer antigen,” as used herein is a molecule or compound (e.g., a protein, peptide, polypeptide, lipid, glycolipid, carbohydrate and/or DNA) associated with a tumour or cancer cell and which is capable of provoking an immune response when expressed on the surface of an antigen presenting cell in the context of an MHC molecule. Cancer antigens include neo-antigens and self-antigens, as well as other antigens that may not be specifically associated with a cancer, but nonetheless induce and/or enhance an immune response to and/or reduce the growth of a tumour or cancer cell when administered to an animal.

An “antigen from a pathogenic and/or infectious organism” as used herein, is an antigen of any organism and includes, but is not limited to, infectious virus (such as influenza or SARS coronaviruses), infectious bacteria, infectious parasites including protozoa (such as Plasmodium sp.) and worms and infectious fungi. Typically, for use in the invention the antigen is a protein or antigenic fragment thereof from the organism, or a synthetic compound which is identical to or similar to naturally-occurring antigen which induces an immune response specific for the corresponding organism. Compounds or antigens that are similar to a naturally-occurring organism antigens are well known to those of ordinary skill in the art. A non-limiting example of a compound that is similar to a naturally-occurring organism antigen is a peptide mimic of a polysaccharide antigen.

Specific embodiments of cancer antigens include, e.g., mutated antigens such as the protein products of the Ras p21 protooncogenes, tumor suppressor p53 and HER-2/neu and BCR-abl oncogenes, as well as CDK4, MUM1, Caspase 8, and Beta catenin; overexpressed antigens such as galectin 4, galectin 9, carbonic anhydrase, Aldolase A, PRAME, Her2/neu, ErbB-2 and KSA, oncofetal antigens such as alpha fetoprotein (AFP), human chorionic gonadotropin (hCG); self-antigens such as carcinoembryonic antigen (CEA) and melanocyte differentiation antigens such as Mart 1/Melan A, gp100, gp75, Tyrosinase, TRP1 and TRP2; prostate associated antigens such as PSA, PAP, PSMA, PSM-PI and PSM-P2; reactivated embryonic gene products such as MAGE 1, MAGE 3, MAGE 4, GAGE 1, GAGE 2, BAGE, RAGE, and other cancer testis antigens such as NY-ESO1, SSX2 and SCP1; mucins such as Muc-1 and Muc-2; gangliosides such as GM2, GD2 and GD3, neutral glycolipids and glycoproteins such as Lewis (y) and globo-H; and glycoproteins such as Tn, Thompson-Freidenreich antigen (TF) and sTn. In one embodiment, the cancer antigen is all or part of Wilms' tumour gene 1 (WT1), preferably all or part of the amino acid sequence shown in SEQ ID NO: 39. In one embodiment, the cancer antigen is all or part of NY-ESO-1, preferably all or part of the amino acid sequence shown in SEQ ID NO: 41.

Cancer antigens and their respective tumor cell targets include, e.g., cytokeratins, particularly cytokeratin 8, 18 and 19, as antigens for carcinoma. Epithelial membrane antigen (EMA), human embryonic antigen (HEA-125), human milk fat globules, MBrI, MBr8, Ber-EP4, 17-IA, C26 and T16 are also known carcinoma antigens. Desmin and muscle-specific actin are antigens of myogenic sarcomas. Placental alkaline phosphatase, beta-human chorionic gonadotropin, and alpha-fetoprotein are antigens of trophoblastic and germ cell tumors. Prostate specific antigen is an antigen of prostatic carcinomas, carcinoembryonic antigen of colon adenocarcinomas. HMB-45 is an antigen of melanomas. In cervical cancer, useful antigens could be encoded by human papilloma virus. Chromogranin-A and synaptophysin are antigens of neuroendocrine and neuroectodermal tumors. Of particular interest are aggressive tumors that form solid tumor masses having necrotic areas.

Antigens derived from pathogens known to predispose to certain cancers may also be advantageously used in the present invention. Pathogens of particular interest for use in the cancer vaccines provided herein include the hepatitis B virus (hepatocellular carcinoma), hepatitis C virus (heptomas), Epstein Barr virus (EBV) (Burkitt lymphoma, nasopharynx cancer, PTLD in immunosuppressed individuals), HTLVL (adult T cell leukemia), oncogenic human papilloma viruses types 16, 18, 33, 45 (adult cervical cancer), and the bacterium Helicobacter pylori (B cell gastric lymphoma). Other medically relevant microorganisms that may serve as antigens in mammals and more particularly humans are described extensively in the literature, e.g., C. G. A Thomas, Medical Microbiology, Bailliere Tindall, (1983).

Exemplary viral pathogens include, but are not limited to, infectious virus that infect mammals, and more particularly humans. Examples of infectious virus, and antigens that can be derived therefrom, include, but are not limited to: Retroviridae (e.g., human immunodeficiency viruses, such as HIV-I (also referred to as HTLV-III, LAV or HTLV-III/LAV, or HIV-III, and other isolates, such as HIV-LP; Picornaviridae (e.g. polio viruses, hepatitis A virus; enteroviruses, human Coxsackie viruses, rhino viruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses such as the SARS coronavirus, SARS-CoV and SARS-CoV-2); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses). In one embodiment, the antigen is from SARS-CoV-2, preferably from the spike protein (e.g. receptor binding domain (RBD; preferably the amino acid sequence shown in SEQ ID NO: 40), 51 subunit only, S2 subunit only, or both 51+S2 subunit antigens). In one embodiment, the antigen is from Influenza A, preferably the extracellular domain of matrix protein 2 (M2e); preferably the amino acid sequence shown in SEQ ID NO: 42.

Also, gram negative and gram positive bacteria may be targeted by the subject compositions and methods in vertebrate animals. Such gram positive bacteria include, but are not limited to Pasteurella sp., Staphylococci sp., and Streptococcus sp. Gram negative bacteria include, but are not limited to, Escherichia coli, Pseudomonas sp., and Salmonella sp. Specific examples of infectious bacteria include but are not limited to: Helicobacter pyloris, Borella burgdorferi, Legionella pneumophilia, Mycobacteria sp. (e.g. M. tuberculosis, M. avium, M. intracellular e, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus infuenzae, Bacillus antracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema per tenue, Leptospira, Rickettsia, and Actinomyces israelii.

Polypeptides of bacterial pathogens which may find use as sources of antigen in the subject compositions include but are not limited to an iron-regulated outer membrane protein, (“IROMP”), an outer membrane protein (“OMP”), and an A-protein of Aeromonis salmonicida which causes furunculosis, p57 protein of Renibacterium salmoninarum which causes bacterial kidney disease (“BKD”), major surface associated antigen (“msa”), a surface expressed cytotoxin (“mpr”), a surface expressed hemolysin (“ish”), and a flagellar antigen of Yersiniosis; an extracellular protein (“ECP”), an iron-regulated outer membrane protein (“IROMP”), and a structural protein of Pasteurellosis; an OMP and a flagellar protein of Vibrosis anguillarum and V. ordalii; a flagellar protein, an OMP protein, aroA, and purA of Edwardsiellosis ictaluri and E. tarda; and surface antigen of Ichthyophthirius, and a structural and regulatory protein of Cytophaga columnari; and a structural and regulatory protein of Rickettsia. Such antigens can be isolated or prepared recombinantly or by any other means known in the art.

Examples of pathogens further include, but are not limited to, infectious fungi and parasites that infect mammals, and more particularly humans. Examples of infectious fungi include, but are not limited to: Cryptococcus neoformans, Histoplasma capsulatum, Coccidioides immitis, Blastomyces dermatitidis, Chlamydia trachomatis, and Candida albicans.

Examples of parasites include intracellular parasites and obligate intracellular 0 parasites. Examples of parasites include but are not limited to Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis, Leishmania tropica, Trypanosoma gambiense, Trypanosoma 5 rhodesiense, Wuchereria bancrofti, Brugia malayi, Brúgia timori, Ascaris lumbricoides, Onchocerca volvulus and Schistosoma mansoni.

Other medically relevant microorganisms that serve as antigens in mammals and more particularly humans are described extensively in the literature, e.g., see C. G. A Thomas, Medical Microbiology, Bailliere Tindall, (1983). In addition to the treatment of infectious human diseases and human pathogens, the compositions and methods of the present invention are useful for treating infections of nonhuman mammals. Exemplary non-human pathogens include, but are not limited to, mouse mammary tumor virus (“MMTV”), Rous sarcoma virus (“RSV”), avian leukemia virus (“ALV”), avian myeloblastosis virus (“AMV”), murine leukemia virus 5 (“MLV”), feline leukemia virus (“FeLV”), murine sarcoma virus (“MSV”), gibbon ape leukemia virus (“GALV”), spleen necrosis virus (“SNV”), reticuloendotheliosis virus (“RV”), simian sarcoma virus (“SSV”), Mason-Pfizer monkey virus (“MPMV”), simian retrovirus type 1 (“SRV-1”), lentiviruses such as HIV-1, HIV-2, SIV, Visna virus, feline immunodeficiency virus (“FIV”), and equine infectious anemia virus 0 (“EIAV”), T-cell leukemia viruses such as HTLV-I , HTLV-II, simian T-cell leukemia virus (“STLV”), and bovine leukemia virus (“BLV”), and foamy viruses such as human foamy virus (“HFV”), simian foamy virus (“SFV”) and bovine foamy virus (“BFV”).

### Protein Production

In one example, an antigen binding protein described herein according to any example is produced by culturing a hybridoma under conditions sufficient to produce the protein, e.g., as described herein and/or as is known in the art.

### Recombinant Expression

In another example, an antigen binding protein, fusion protein or conjugate described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), a-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHOS promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's FIO (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Methods for isolating a protein are known in the art and/or described herein.

Where an antigen binding protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The antigen binding protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

### Assaying Activity of an Antigen Binding Protein

### Binding to CLEC9A and Mutants Thereof

It will be apparent to the skilled artisan from the disclosure herein that antigen binding proteins of the present invention bind to CLEC9A. Methods for assessing binding to a protein are known in the art, e.g., as described in Scopes (In: Protein purification: principles and practice, Third Edition, Springer Verlag, 1994). Such a method generally involves immobilizing the antigen binding protein and contacting it with labeled antigen (CLEC9A). Following washing to remove non-specific bound protein, the amount of label and, as a consequence, bound antigen is detected. Of course, the antigen binding protein can be labeled and the antigen immobilized. Panning-type assays can also be used. Alternatively, or additionally, surface plasmon resonance assays can be used.

Optionally, the dissociation constant (Kd), association constant (Ka) and/or affinity constant (KD) of an immobilized antigen binding protein for CLEC9A or an epitope thereof is determined. The “Kd” or “Ka” or “KD” for a CLEC9A-binding protein is in one example measured by a radiolabeled or fluorescently-labeled CLEC9A ligand binding assay. In the case of a “Kd”, this assay equilibrates the antigen binding protein with a minimal concentration of labeled CLEC9A or epitope thereof in the presence of a titration series of unlabeled CLEC9A. Following washing to remove unbound CLEC9A or epitope thereof, the amount of label is determined, which is indicative of the Kd of the protein.

According to another example the Kd, Ka or KD is measured by using surface plasmon resonance assays, e.g., using BlAcore surface plasmon resonance (BlAcore, Inc., Piscataway, NJ) with immobilized CLEC9A or a region thereof or immobilized antigen binding protein.

Typically, the antigen binding protein binds to CLEC9A but does not detectably activate dendritic cells.

### Compositions

In some examples, an antigen binding protein, fusion protein, conjugate or pharmaceutical composition as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing an antigen binding protein, fusion protein or conjugate into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for parenteral administration, such as intravenous administration or administration into a body cavity or lumen of an organ or joint. The compositions for administration will commonly comprise a solution of an antigen binding protein dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding protein of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

Upon formulation, an antigen binding protein of the present invention will be administered in a manner compatible with the dosage formulation and in such amount as is therapeutically/prophylactically effective. Formulations are easily administered in a variety of dosage forms, such as the type of injectable solutions described above, but other pharmaceutically acceptable forms are also contemplated, e.g., tablets, pills, capsules or other solids for oral administration, suppositories, pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver an antigen binding protein of the present invention.

WO2002/080967 describes compositions and methods for administering aerosolized compositions comprising antibodies for the treatment of, e.g., asthma, which are also suitable for administration of an antigen binding protein of the present invention.

### Dosages and Timing of Administration

Suitable dosages of an antigen binding protein of the present invention will vary depending on the specific an antigen binding protein, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED50 of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC50 (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present invention comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding protein(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific antigen binding protein(s), fusion protein(s) or conjugate(s) administered and/or the particular subject and/or the type or severity or level of condition and/or predisposition (genetic or otherwise) to the condition. Accordingly, this term is not to be construed to limit the present invention to a specific quantity, e.g., weight or amount of antigen binding protein(s), rather the present invention encompasses any amount of the antigen binding protein(s) sufficient to achieve the stated result in a subject.

### Kits

The present invention additionally comprises a kit comprising one or more of the following:
(i) an antigen binding protein, fusion protein or conjugate of the invention or expression construct(s) encoding same;
(ii) a cell of the invention;
(iii) a complex of the invention; or
(iii) a pharmaceutical composition of the invention.

In the case of a kit for detecting CLEC9A, the kit can additionally comprise a detection means, e.g., linked to an antigen binding protein of the invention.

In the case of a kit for therapeutic/prophylactic use, the kit can additionally comprise a pharmaceutically acceptable carrier.

Optionally a kit of the invention is packaged with instructions for use in a method described herein according to any example.

The present invention also provides a kit as described herein when used in a method of the invention.

Summary of amino acid and nucleotide sequences

| Antibody or | | SEQ | |
|---|---|---|---|
| Antigen ID | Region | ID NO: | Amino acid or nucleotide sequence |
| Ab1 | CDR L1 | 1 | QSLLHSDGNTY |
| (Humanised | (protein) | | |
| CLEC9A | | | |
| Ab) | | | |
| | CDR L2 | 2 | RIS |
| | (protein) | | |
| | CDR L3 | 3 | LQSSHFPPT |
| | (protein) | | |
| | CDR H1 | 4 | GFTFNNYW |
| | (protein) | | |
| | CDR H2 | 5 | ITTAAGGT |
| | (protein) | | |
| | CDR H3 | 6 | TRVGRDIWDY |
| | (protein) | | |
| | VL | 7 | DIVMTQTPLSLSVTPGQPASISCKSSQSLLHSDGNTYLHWYLQKPGQPPQLLIW |
| | (protein) | | RISNRFSGVPDRFSGSGSGTDFTLKISRVEAEDVGVYYCLQSSHFPPTFGGGTK |
| | | | VEIKR |
| | VH | 8 | EVQLLESGGGLVQPGGSLRLSCAASGFTFNNYWLAWVRQAPGKGLEWVASITTA |
| | (protein) | | AGGTYYPDSVKGRFTISRDNSKNTLYLQMNSLRAEDTAVYYCTRVGRDIWDYWG |
| | | | QGTLVTVSS |
| | CDR L1 | 9 | cagagcctgctgcacagcgacggcaatacctac |
| | (DNA) | | |
| | CDR L2 | 10 | cggatcagc |
| | (DNA) | | |
| | CDR L3 | 11 | ctgcagagcagccactttcctccaacc |
| | (DNA) | | |
| | CDR H1 | 12 | ggcttcaccttcaacaattattgg |
| | (DNA) | | |
| | CDR H2 | 13 | attaccaccgctgccggcggaacc |
| | (DNA) | | |
| | CDR H3 | 14 | accagagtgggcagagacatctgggattac |
| | (DNA) | | |
| | VL (DNA) | 15 | GACATCGTGATGACCCAAACACCTCTGAGCCTGAGCGTGACACCTGGACAGCCT |
| | | | GCCAGCATCAGCTGTAAAAGCAGCCAGAGCCTGCTGCACAGCGACGGCAATACC |
| | | | TACCTGCACTGGTATCTGCAGAAGCCCGGACAGCCTCCTCAGCTGCTGATTTGG |
| | | | CGGATCAGCAACAGATTCAGCGGCGTGCCCGATAGATTTTCTGGCTCTGGCAGC |
| | | | GGCACCGACTTCACCCTGAAGATTAGCAGAGTGGAAGCCGAGGACGTGGGCGTG |
| | | | TACTACTGTCTGCAGAGCAGCCACTTTCCTCCAACCTTTGGCGGAGGCACCAAG |
| | | | GTGGAAATCAAGAGA |
| | VH (DNA) | 16 | GAGGTGCAGCTGCTTGAATCTGGCGGAGGACTTGTTCAGCCTGGCGGCTCTCTG |
| | | | AGACTGTCTTGTGCCGCCAGCGGCTTCACCTTCAACAATTATTGGCTGGCCTGG |
| | | | GTCCGACAGGCCCCTGGAAAAGGACTTGAGTGGGTCGCCTCTATTACCACCGCT |
| | | | GCCGGCGGAACCTACTATCCCGATTCTGTGAAGGGCAGATTCACCATCAGCCGG |
| | | | GACAACAGCAAGAACACCCTGTACCTGCAGATGAACAGCCTGAGAGCCGAGGAC |
| | | | ACCGCCGTGTACTACTGTACCAGAGTGGGCAGAGACATCTGGGATTACTGGGGC |
| | | | CAGGGCACCCTGGTTACAGTGTCTAGC |
| | FR L1 | 17 | DIVMTQTPLSLSVTPGQPASISCKSS |
| | (protein) | | |
| | FR L2 | 18 | LHWYLQKPGQPPQLLIW |
| | (protein) | | |
| | FR L3 | 19 | NRFSGVPDRFSGSGSGTDFTLKISRVEAEDVGVYYC |
| | (protein) | | |
| | FR L4 | 20 | FGGGTKVEIKR |
| | (protein) | | |
| | FR H1 | 21 | EVQLLESGGGLVQPGGSLRLSCAAS |
| | (protein) | | |
| | FR H2 | 22 | LAWVRQAPGKGLEWVAS |
| | (protein) | | |
| | FR H3 | 23 | YYPDSVKGRFTISRDNSKNTLYLQMNSLRAEDTAVYYC |
| | (protein) | | |
| | FR H4 | 24 | WGQGTLVTVSS |
| | (protein) | | |
| | FR L1 | 25 | gacatcgtgatgacccaaacacctctgagcctgagcgtgacacctggacagcct |
| | (DNA) | | gccagcatcagctgtaaaagcagc |
| | FR L2 | 26 | ctgcactggtatctgcagaagcccggacagcctcctcagctgctgatttgg |
| | (DNA) | | |
| | FR L3 | 27 | aacagattcagcggcgtgcccgatagattttctggctctggcagcggcaccgac |
| | (DNA) | | ttcaccctgaagattagcagagtggaagccgaggacgtgggcgtgtactactgt |
| | FR L4 | 28 | tttggcggaggcaccaaggtggaaatcaagaga |
| | (DNA) | | |
| | FR J1 | 29 | gaggtgcagctgcttgaatctggcggaggacttgttcagcctggcggctctctg |
| | (DNA) | | agactgtcttgtgccgccagc |
| | FR H2 | 30 | ctggcctgggtccgacaggcccctggaaaaggacttgagtgggtcgcctct |
| | (DNA) | | |
| | FR H3 | 31 | tactatcccgattctgtgaagggcagattcaccatcagccgggacaacagcaag |
| | (DNA) | | aacaccctgtacctgcagatgaacagcctgagagccgaggacaccgccgtgtac |
| | | | tactgt |
| | FR H4 | 32 | TGGGGCCAGGGCACCCTGGTTACAGTGTCTAGC |
| | (DNA) | | |
| | Heavy | 33 | MDIRLSLVFLVLFIKGVQCEVQLLESGGGLVQPGGSLRLSCAASGFTFNNYWLA |
| | chain | | WVRQAPGKGLEWVASITTAAGGTYYPDSVKGRFTISRDNSKNTLYLQMNSLRAE |
| | | | DTAVYYCTRVGRDIWDYWGQGTLVTVSSASTKGPSVFPLAPCSRSTSESTAALG |
| | | | CLVKDYFPEPVTVSWNSGALTSGVHTFPAVLQSSLGYSLSSVVTVPSSSLGTKT |
| | | | YTCNVDHKPSNTKVDKRVESKYGPPCPPCPAPEFEGGPSVFLFPPKPKDTLMIS |
| | | | RTPEVTCVVVDVSQEDPEVQFNWYVDGVEVHNAKTKPREEQFNSTYRVVSVLTV |
| | | | LHQDWLNGKEYKCKVSNKGLPSSIEKTISKAKGQPREPQVYTLPPSQEEMTKNQ |
| | | | VSLTCLVKGFYPSDIAVEWESNGQPENNYKTTPPVLDSDGSFFLYSRLTVDKSR |
| | | | WQEGNVFSCSVMHEALHNHYTQKSLSLSLGK |
| | | | Predicted leader in italics, human constant regions |
| | | | IgG4 underlined only, CDRs according to IMGT bold and |
| | | | underlined. Single amino acids in bold in the constant |
| | | | regions are point mutations to stabilise the hinge (P) |
| | | | and stop FcR binding (E). |
| | Light | 34 | MKWPVRLLVLFFWIPVSRGDIVMTQTPLSLSVTPGQPASISCKSSQSLLHSDGN |
| | chain | | TYLHWYLQKPGQPPQLLIWRISNRFSGVPDRFSGSGSGTDFTLKISRVEAEDVG |
| | | | VYYCLQSSHFPPTFGGGTKVEIKRTVAAPSVFIFPPSDEQLKSGTASVVCLLNN |
| | | | FYPREAKVQWKVDNALQSGNSQESVTEQDSKDSTYSLSSTLTLSKADYEKHKVY |
| | | | ACEVTHQGLSSPVTKSFNRGEC |
| | | | Predicted leader in italics, human constant regions |
| | | | IgG4 underlined only, CDRs according to IMGT bold and |
| | | | underlined. |
| CLEC9A | | 35 | RWLWQDGSSPSPGLLPAERSQSANQVC |
| epitope | | | |
| Full length | | 36 | MHEEEIYTSLQWDSPAPDTYQKCLSSNKCSGACCLVMVISCVFCMGLLTASIFL |
| human | | | GVKLLQVSTIAMQQQEKLIQQERALLNFTEWKRSCALQMKYCQAFMQNSLSSAH |
| CLEC9A | | | NSSPCPNNWIQNRESCYYVSEIWSIWHTSQENCLKEGSTLLQIESKEEMDFITG |
| | | | SLRKIKGSYDYWGLSQDGHSGRWLWQDGSSPSPGLLPAERSQSANQVCGYVKSN |
| | | | SLLSSNCSTWKYFICEKYALRSSV |
| VL from rat | VL | 37 | EILMTQTPVSLPVSLGGQVSFSCRSSQSLLHSDGNTYLHWYLQKPGQSPQLLIW |
| anti- | (protein) | | RISNRFSGVPDRFSGSGSGTDFTLKISRVEPEDLGVYYCLQSSHFPPTFGGGTN |
| CLEC9A | | | LELKR |
| antibody | | | |
| VH from rat | VH | 38 | EVQLVESGGGLVQPGGSLKLSCVASGFTFNNYWLAWIRQAPRKGLEWVASITTA |
| anti-CLEC9A | (protein) | | AGGTYYPDSVKGRFTISRDNAKSTLYLQMNSLTSEDTATYYCTRVGRDIWDYWG |
| antibody (or | | | QGVVVTVSS |
| human/rat | | | |
| chimeric | | | |
| anti-CLEC9A | | | |
| antibody) | | | |
| WT1 | | 39 | SSGQARMFPNAPYLPSSQLECMTWNQMNLGACNKRYFKLSHLQMHSRKHTG |
| antigen | | | |
| SARS-COV-2 | | 40 | KSFTVEKGIYQTSNFRVQPTESIVRFPNITNLCPFGEVFNATRFASVYAWNRKR |
| RBD | | | ISNCVADYSVLYNSASFSTFKCYGVSPTKLNDLCFTNVYADSFVIRGDEVRQIA |
| antigen | | | PGQTGKIADYNYKLPDDFTGCVIAWNSNNLDSKVGGNYNYLYRLFRKSNLKPFE |
| | | | RDISTEIYQAGSTPCNGVEGFNCYFPLQSYGFQPTNGVGYQPYRVVVLSFELLH |
| | | | APATVCGPKKSTNLVKNKCVNFNFNGLTGTG |
| NY-ESO-1 | | 41 | LLEFYLAMPFATPMEAELARRSLAQDAPPLPVPGVLLKEFTVSGNILTIRLTAA |
| antigen | | | DHRQLQLSISSCLQQLSLLMWITQCFLPVFLAQPPSGQRR |
| M2e antigen | | 42 | MSLLTEVETPIRNEWGCRCNDSSDGGGMSLLTEVETPIRNEWGCRCNDSSDGGG |
| | | | MSLLTEVETPIRNEWGCRCNDSSDGGG |
| VL from | VL | 43 | EILMTQTPVSLPVSLGGQVSFSCRSSQSLLHSDGNTYLHWYLQKPGQSPQLLIW |
| human/rat | (protein) | | RISNRFSGVPDRFSGSGSGTDFTLKISRVEPEDLGVYYCLQSSHFPPTFGGGTN |
| chimeric | | | LEIKR |
| anti-CLEC9A | | | |
| antibody | | | |

### EXAMPLES

### Example 1-Design of Humanised Anti-CLEC9A Antibody

Constructs encoding the humanised anti-CLEC9A Ab (Ab 1 as per Table 1 above) were generated based on the sequences encoding human chimeric anti-Clec9A Ab (clone 4C6), with human IgG4 and kappa constant regions and 2 point mutations in the IgG4 constant region to abrogate FcR binding and stabilise disulphide bonds—P at position 244 (often referred to as S246P) is a mutation to stabilise the hinge and E at position 251(often referred to as L253E) is a mutation to stop FcR binding.

Humanized Ab 1 was generated by transferring the CDRs of the 4C6 mAb (CDR-H1, CDR-H2, CDR-H3, CDR-L1, CDR-L2, and CDR-L3) onto human framework regions using standard molecular techniques. IMGTN-QUEST and IMGT/Junctions analysis tools were used to identify human germline genes in which sequences from the variable regions of both the heavy and light chains were closely aligned with those of rat antibody. Framework sequences of these selected human germline genes were used as acceptor sequences for the 4C6 CDRs (IGHV3-23*01 and IGKV2D-29*01 human genes according to IMGT database). However, rat residues were retained in the critical “Vernier” zone. The humanized VH and VL DNA genes, which were also codon optimized for expressed in HEK cells, were synthesized by GeneArt.

### Example 2-Production of Humanised Anti-CLEC9A Antibody

Purification: Freestyle 293F cells were transfected with constructs encoding the heavy and light chains of humanised anti-CLEC9A Ab (Ab1 comvec12: IgG4 and kappa), or heavy chains of anti-CLEC9A Ab genetically fused to tumour associated antigen WT1 (SEQ ID No; 39), tumour associated antigen NY-ESO1 (SEQ ID No 41), SARS-CoV2-RBD (SEQ ID No: 40) and Influenza M2e (SEQ ID No 42) antigens. Ab was purified from the culture supernatant 6 days post transfection on Protein A using binding buffer (1.5 M glycine, 3 M NaCl pH8.9 with NaOH), and elution either with acetic acid pH3.5 buffers (150 mM NaCl, 100 mM acetic acid pH3.5) or with a citric acid pH3 buffer (100 mM citric acid pH3 with NaOH). Purified Ab were dialysed against PBS pH7.4.

ELISA: Plates were coated with soluble human CLEC9A (1 μg/ml) or soluble mouse CLEC12A (1 μg/ml). Anti-CLEC9A Ab (0.1 μg/ml), anti-CLEC9A Ab-Ag (0.2 μg/ml) were added and titrated down the ELISA plate. Bound Ab was detected with anti-human IgG4-biotin and streptavidin HRP. ELISA plates were visualised with ABTS at 405 nm absorbance, subtracting background at 490 nm.

Binding to 293F cells: 293F cells were transfected with construct encoding full-length human CLEC9A and were stained with 5, 2.5, 1.25 and 0.625 μg/ml humanised anti-CLEC9A-Ab or anti-CLEC9A-Ab-Ag 24h post transfection. Bound Ab was detected with anti-human IgG4-biotin and streptavidin-PE, on live cells. Dead cells were excluded with propidium iodide or live/dead-Aqua.

Binding to blood DC: PBMC were isolated whole blood by Ficoll-Plaque Plus density centrifugation. DC were enriched with the human pan-DC enrichment kit (Stemcell technologies), blocked with Fc block (BD Biosciences) and stained with anti-CLEC9A-Ab, anti-CLEC9A-Ab-Ag or isotype control Ab (10 μg/ml), CD14, CD16, CD19, HLA-DR, CD123, CD141, CD1c, and live/dead aqua to exclude dead cells. Anti-CLEC9A-Ab binding was detected with anti-human IgG4-biotin and streptavidin-PE.

Constructs encoding the humanised anti-CLEC9A Ab were generated from the human/rat chimaeric anti-CLEC9A Ab (clone 4C6, Tullett et al., 2016), with human IgG4 and kappa constant regions and 2 point mutations in the IgG4 constant region to abrogate FcR binding and stabilise disulphide bonds. Humanised anti-Clec9A Ab were expressed using a mammalian expression system (Freestyle 293F cells), and purified using Protein A, with a yield of 85.5 mg/L.

### Example 3-Validation of Humanised Anti-CLEC9A Antibody Binding and Specificity

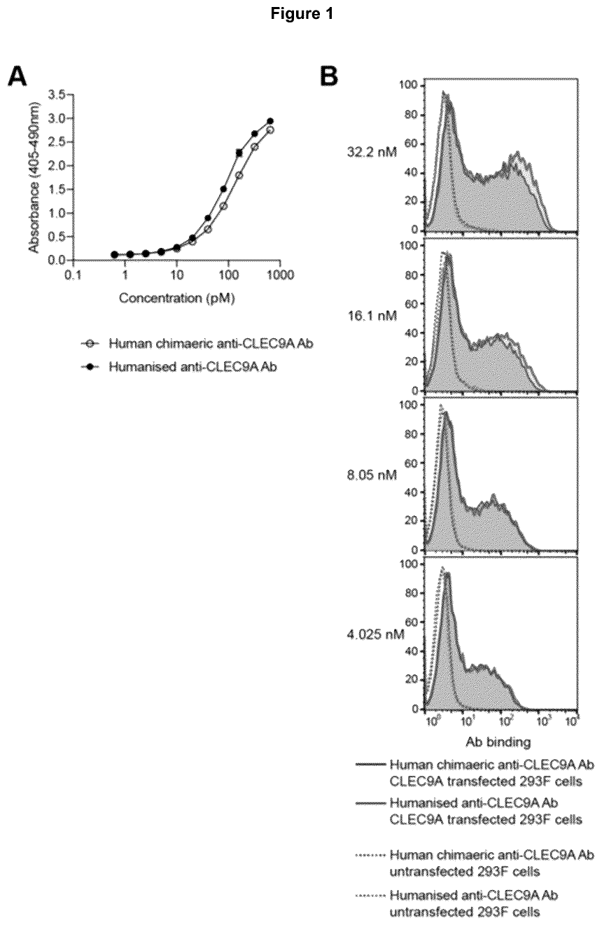

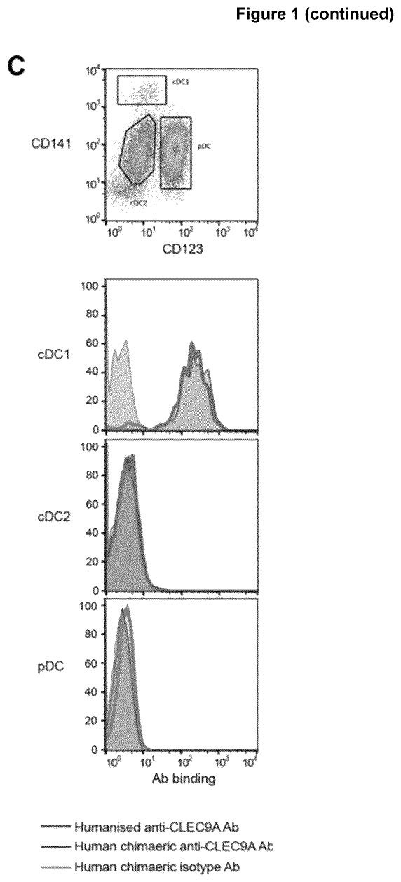

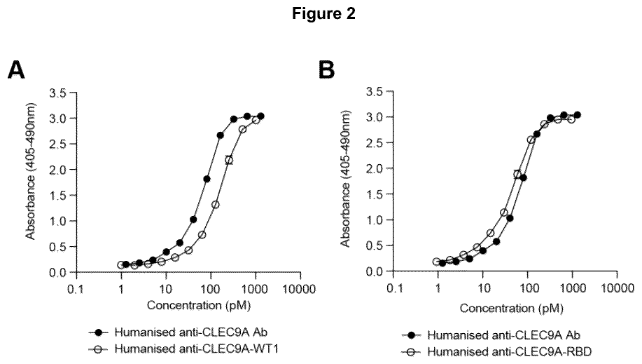

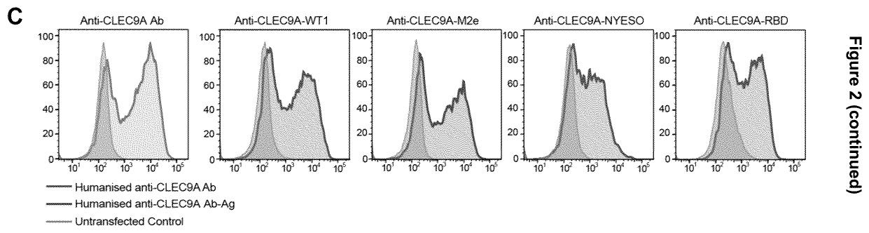

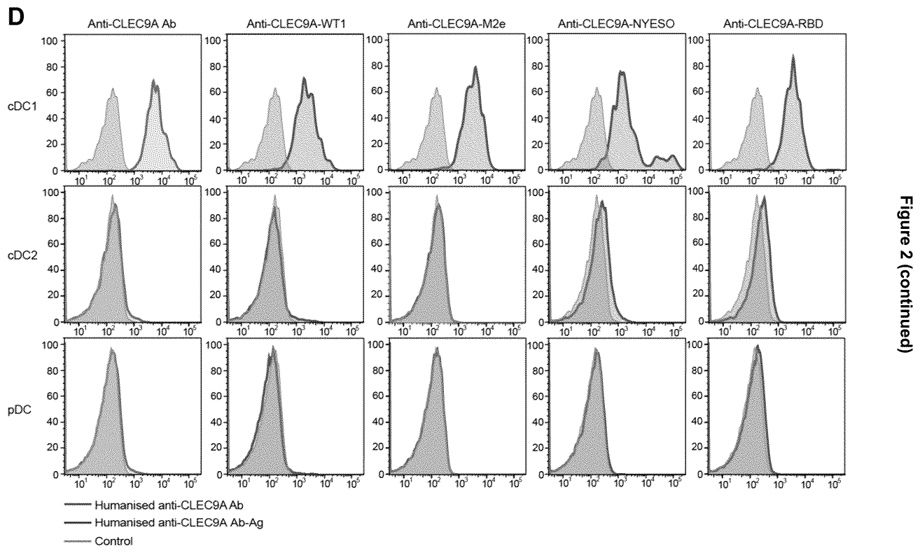

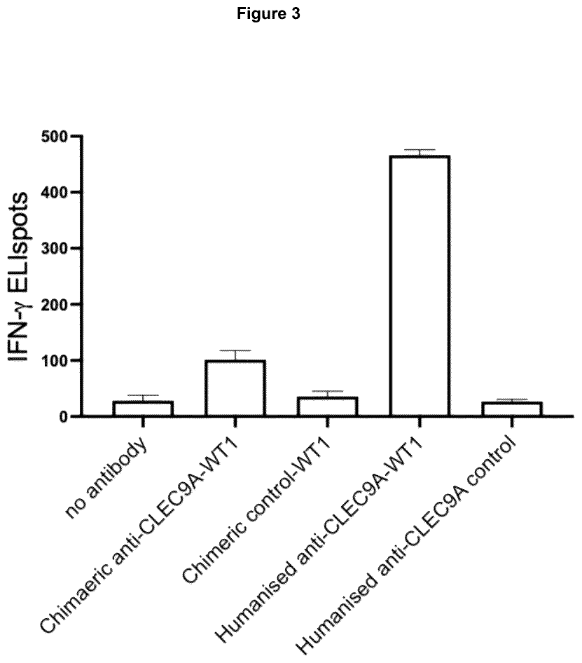

The inventors next validated the binding of the humanised anti-CLEC9A Ab. The humanised anti-CLEC9A Ab and the original human chimaeric anti-CLEC9A Ab bound comparably to soluble CLEC9A by ELISA (FIG. 1A) and to cell surface CLEC9A on transfected 293F cells by flow cytometric analysis (FIG. 1B). The inventors further confirmed that the humanised anti-CLEC9A Ab bound specifically to human blood cDC1, but not to cDC2 or pDC (FIG. 1C). The isotype control did not bind to any DC subset.

### Example 4-Design and Production of Antibody-Antigen Fusion Proteins

The inventors also generated humanised anti-CLEC9A Ab carrying antigens derived from the tumour antigens WT1 and NY-ESO-1, and to infectious disease vaccine candidate antigens SARS-CoV-2 antigen receptor binding domain (RBD) from the Spike protein, and Influenza M2e. In brief, constructs encoding the heavy chain of humanised anti-CLEC9A Ab genetically fused to antigenic sequences of WT1, NY-ESO-1, RBD, and M2e were generated. Humanised anti-CLEC9A and anti-CLEC9A Ab-Ag were expressed in 293F cells, and shown to bind to CLEC9A expressed on the surface of transfected 293F cells (FIG. 2).

Humanised anti-CLEC9A Ab-carrying WT1, NY-ESO1, RBD and M2e were subsequently transfected and purified from large scale 293F culture supernatant on Protein A Humanised anti-CLEC9A Ab-Ag constructs were validated using binding studies including CLEC9A expressed on the surface of CLEC9A-transfected cells by flow cytometry (FIG. 2C), or to soluble CLEC9A by ELISA (FIG. 2A, B). Anti-CLEC9A Ab-Ag have been further validated using human blood DC, and shown to bind to human blood cDC1, but not to cDC2 or pDC (FIG. 2D).

### Example 5 -Assessing Activation of WT1 Specific CD8+T Cells

Humanised anti-CLEC9A Ab-carrying WT1 have also been shown to activate WT1-specific T cells, demonstrating the efficacy of humanised anti-CLEC9AAb for Ag delivery to DC and for immune modulation (FIG. 3).

Generation of humanised mice that develop human naïve WT1₂₃₅₋₂₄₃-specific CD8+ T cells and human cDC1 dendritic cells: Human HLA-A*2402+CD34+ hematopoeitic progenitor cells were isolated from cord blood using a CD34+ cell isolation kit (Miltenyi Biotec) and transduced with a lentivirus encoding a T cell receptor (TCR) specific for the HLA-A*2402-restricted WT1 ₂₃₅₋₂₄₃ peptide epitope. Two to five day old NSG-A24 (NOD.Cg-PrkdcscidIL2rgtm1Whl Tg (HLA-A24/H2-D/B2M) 3Dvs/Sz) mice were irradiated (10 Gy) followed by intrahepatic injection of the transduced human CD34+ progenitor cells. Reconstitution of a human immune system (“humanised mice”) was confirmed at 10-14 weeks by detection of human CD45+ cells in blood. Human cDC1 were expanded in vivo by subcutaneous administration of Flt3L (Bio-X Cell, West Lebanon, NH, USA; 2×50 μg 4 days apart).

Activation of WT1-specific CD8+ T cell responses: Spleens from humanised mice were harvested 10 days after the second Flt3L administration, digested in collagenase IV (Worthington Biochemical) and DNase I (Roche), separated over a Percoll density gradient, and enriched for human leukocytes using a Mouse/Human Chimera EasySep Kit (Stemcell Technologies). The presence of human CD8+ T cells expressing the HLA-A*2402-restricted WT1₂₃₅₋₂₄₃ TCR was confirmed by flow cytometry after staining with the corresponding tetramer conjugated to Allophycocyanin (APC), followed by anti-rat CD2-PE (clone OX-34) and the anti-human Abs anti-CD45-BUV395 (clone Hl30, BD Biosciences), anti-CD3-Pacific Blue or anti-CD3-BV711 (clone OKT3), and anti-CD8-PE-Cy7 (clone RPA-T8).

In order to assess T cell activation, the human leukocytes were incubated overnight with chimeric or humanised anti-CLEC9A-WT1 antibodies or negative controls (no antibody, chimeric control-WT1 or humanized anti-CLEC9A control) at 10 μg/mL in culture medium RPMI 1640 medium (Gibco) supplemented with 10% FBS, HEPES (10 mM), sodium pyruvate (1 mM), penicillin/streptomycin (100 U/mL), GlutaMAX (2 mM), non-essential amino acids (0.1 mM) (all from Life Technologies), and 2-mercaptoethanol (50 μM, Sigma-Aldrich).

Interferon-γ (IFNγ) production by T cells in response to humanised anti-CLEC9A-WT1 was compared to human chimeric anti-CLEC9A-WT1, human chimeric control-WT1 (anti-β-galactosidase-WT1 as irrelevant antibody control with WT1), and humanised anti-CLEC9A (anti-CLEC9A antibody control with no WT1 peptide). Splenocytes treated with controls (no antigen, chimeric control-WT1, humanised anti-CLEC9A control) activated relatively few WT1 specific CD8+ T cells whereas both the human/rat chimeric anti-CLEC9A-WT1, and humanised anti-CLEC9A-WT1 induced substantial IFNγ production. The humanised anti-CLEC9A-WT1 induced significantly more IFNγ production when compared with the chimeric rat anti-human CLEC9A-WT1 at the same concentration (p<0.0001, FIG. 3).

It will be understood that the invention disclosed and defined in this specification extends to all alternative combinations of two or more of the individual features mentioned or evident from the text or drawings. All of these different combinations constitute various alternative aspects of the invention.

1. An antigen binding protein that binds to or specifically binds to CLEC9A, wherein the antigen binding protein comprises:
(a) a framework region (FR) 1 comprising a sequence at least about 58%, at least about 60%, at least 65%, at least 70%, at least 75%, at least 80%, at least about 85%, at least 90%, at least 95% identical to a sequence set forth in SEQ ID NO: 17, a FR2 comprising a sequence at least about 95% identical to a sequence set forth in SEQ ID NO: 18, a FR3 comprising a sequence at least about 95% identical to a sequence set forth in SEQ ID NO: 19, and a FR4 comprising a sequence at least about 73%, at least about 75%, at least 80%, at least 85%, at least 90%, at least 95% identical to a sequence set forth in SEQ ID NO: 20; or
(b) a FR1 comprising a sequence at least about 88%, at least 90%, at least 95% identical to a sequence set forth in SEQ ID NO: 21, a FR2 comprising a sequence at least about 88%, at least 90%, at least 95% identical to a sequence set forth in SEQ ID NO: 22, a FR3 comprising a sequence at least about 87%, at least 90%, at least 95% identical to a sequence set forth in SEQ ID NO: 23, and a FR4 comprising a sequence at least about 82%, at least 85%, at least 90%, at least 95% identical to a sequence set forth in SEQ ID NO: 24.

2. An antigen binding protein according to claim 1, wherein the antigen binding protein comprises:
(c) a framework region (FR) 1 comprising an amino acid sequence with 1, 2, 3, 4, 5, 6, 7, 8, 9 or 10 sequence differences compared to the amino acid set forth in SEQ ID NO: 17, a FR2 comprising an amino acid sequence with 0 amino acid differences compared to the amino acid sequence set forth in SEQ ID NO: 18, a FR3 comprising an amino acid sequence with 1 sequence difference compared to the amino acid sequence set forth in SEQ ID NO: 19, and a FR4 comprising an amino acid sequence with 1 or 2 sequence differences compared to the amino acid sequence set forth in SEQ ID NO: 20; or
(d) a FR1 comprising an amino acid sequence with 1 or 2 sequence differences compared to the amino acid set forth in SEQ ID NO: 21, a FR2 comprising an amino acid sequence with 1 amino acid difference compared to the amino acid sequence set forth in SEQ ID NO: 22, a FR3 comprising an amino acid sequence with 1, 2, 3 or 4 sequence differences compared to the amino acid sequence set forth in SEQ ID NO: 23, and a FR4 comprising an amino acid sequence 1 sequence difference compared to the amino acid sequence set forth in SEQ ID NO: 24.

3. An antigen binding protein according to claim 1, wherein the antigen binding protein comprises each of (a) and (b).

4. An antigen binding protein according to claim 2, wherein the antigen binding protein comprises each of (c) and (d).

5. An antigen binding protein according to any one of claims 1 to 4, wherein the antigen binding protein binds to or specifically binds to an epitope of CLEC9A comprising or consisting of the sequence of SEQ ID NO: 35.

6. An antigen binding protein according to any one of claims 1 to 5, wherein the antigen binding protein comprises:
(e) framework regions comprising each of the sequences set forth in SEQ ID Nos: 17 to 20; or
(f) framework regions comprising each of the sequences set forth in SEQ ID Nos: 21 to 24.

7. An antigen binding protein according to any one of claims 1 to 6, wherein the framework regions comprise each of the sequences set forth in SEQ ID Nos: 17 to 24.

8. An antigen binding protein according to any one of claims 1 to 7, wherein the antigen binding protein comprises complementarity determining regions (CDR), wherein:
CDR1 has a sequence of: QSLLHSDGNTY (SEQ ID NO: 1), or a sequence with 1, 2, 3 or 4 sequence differences compared to the amino acid sequence of SEQ ID NO: 1,
CDR2 has a sequence of: RIS (SEQ ID NO: 2), or a sequence with 1 or 2 sequence differences compared to the amino acid sequence of SEQ ID NO: 2, and
CDR3 has a sequence of: LQSSHFPPT (SEQ ID NO: 3), or a sequence with 1, 2, 3 or 4 sequence differences compared to the amino acid sequence of SEQ ID NO: 3.

9. An antigen binding protein according to any one of claims 1 to 7, wherein the antigen binding protein comprises complementarity determining regions (CDR), wherein:
CDR1 has a sequence of: QSLLHSDGNTY (SEQ ID NO: 1),
CDR2 has a sequence of: RIS (SEQ ID NO: 2), and
CDR3 has a sequence of: LQSSHFPPT (SEQ ID NO: 3).

10. An antigen binding according to any one of claims 1 to 9, wherein the antigen binding protein comprises complementarity determining regions, wherein:
CDR1 has a sequence of: GFTFNNYW (SEQ ID NO: 4) or a sequence with 1, 2, 3 or 4 sequence differences compared to the amino acid sequence of SEQ ID NO: 4,
CDR2 has a sequence of: ITTAAGGT (SEQ ID NO: 5) or a sequence with 1, 2, 3 or 4 sequence differences compared to the amino acid sequence of SEQ ID NO: 5, and
CDR3 has a sequence of: or TRVGRDIWDY (SEQ ID NO: 6) or a sequence with 1, 2, 3 or 4 sequence differences compared to the amino acid sequence of SEQ ID NO: 6.

11. An antigen binding according to any one of claims 1 to 9, wherein the antigen binding protein comprises complementarity determining regions, wherein:
CDR1 has a sequence of: GFTFNNYW (SEQ ID NO: 4),
CDR2 has a sequence of: ITTAAGGT (SEQ ID NO: 5), and
CDR3 has a sequence of: or TRVGRDIWDY (SEQ ID NO: 6).

12. An antigen binding protein according to any one of claims 1 to 11, wherein the antigen binding protein comprises the sequence set forth in SEQ ID NO: 7.

13. An antigen binding protein according to any one of claims 1 to 12, wherein the antigen binding protein comprises the sequence set forth in SEQ ID NO: 8.

14. An antigen binding protein according to any one of claims 1 to 13, wherein the antigen binding protein comprises the sequences set forth in SEQ ID NO: 7 and 8.

15. An antigen binding protein according to any one of claims 1 to 14, wherein the antigen binding protein comprises the sequence set forth in SEQ ID NO: 33.

16. An antigen binding protein according to any one of claims 1 to 15, wherein the antigen binding protein comprises the sequence set forth in SEQ ID NO: 34.

17. An antigen binding protein according to any one of claims 1 to 16, wherein the antigen binding protein comprises the sequence set forth in SEQ ID NO: 33 and 34.

18. An antigen binding protein according to any one of claims 1 to 17, wherein the antigen binding protein is in the form of:
(i) a single chain Fv fragment (scFv);
(ii) a dimeric scFv (di-scFv);
(iii) one of (i) or (ii) linked to a constant region of an antibody, Fc or a heavy chain constant domain (CH) 2 and/or CH3, or
(iv) one of (i) or (ii) linked to a protein that binds to an immune effector cell.

19. An antigen binding protein according to any one of claims 1 to 17, wherein the antigen binding protein is in the form of:
(i) a diabody;
(ii) a triabody;
(iii) a tetrabody;
(iv) a bi-specific antibody;
(v) a Fab;
(vi) a F(ab′)2;
(vii) a Fv;
(viii) one of (i) to (vii) linked to a constant region of an antibody, Fc or a heavy chain constant domain (CH) 2 and/or CH3,
(ix) one of (i) to (vii) linked to a protein that binds to an immune effector cell.

20. An antigen binding protein according to any one of claims 1 to 19, wherein the antigen binding protein is a monoclonal antibody.

21. A fusion protein comprising an antigen binding protein according to any one of claims 1 to 20.

22. A fusion protein according to claim 21, wherein the fusion protein further comprises an antigen.

23. A conjugate in the form of an antigen binding protein according to any one of claims 1 to 20 or fusion protein according to claim 21 or 22 conjugated to a label or a therapeutic agent.

24. A conjugate according to claim 23, wherein the therapeutic agent is an antigen, a cytotoxic agent, a drug and/or pharmacological agent.

25. A fusion protein according to claim 21 or 22, or a conjugate according to claim 23 or 24, wherein the antigen is a cancer antigen, a self-antigen, an allergen, and/or an antigen from a pathogenic and/or infectious organism.

26. A fusion protein or conjugate according to claim 25, wherein the pathogenic and/or infectious organism is a virus or bacterium.

27. A fusion protein according to claim 21 or 22, or a conjugate according to claim 23 or 24, wherein the antigen is WT-1 or NY-ESO-1 or a fragment thereof.

28. A fusion protein according to claim 21 or 22, or a conjugate according to claim 23 or 24, wherein the antigen is from SARS-CoV-2.

29. A fusion protein according to claim 21 or 22, or a conjugate according to claim 23 or 24, wherein the antigen is the RBD from SARS-CoV-2 or a fragment thereof.

30. A fusion protein according to claim 21 or 22, or a conjugate according to claim 23 or 24, wherein the antigen is from influenza.

31. A nucleic acid encoding an antigen binding protein, fusion protein or conjugate of any one of claims 1 to 30, preferably wherein the nucleic acid is mRNA.

32. A vector comprising the nucleic acid according to claim 31, preferably wherein the vector is a viral vector or non-viral vector.

33. A cell comprising a vector according to claim 32 or nucleic acid according to claim 31.

34. A pharmaceutical composition comprising an antigen binding protein, fusion protein, conjugate, nucleic acid, vector or cell according to any one of claims 1 to 33 and a pharmaceutically acceptable carrier, diluent or excipient.

35. A pharmaceutical composition according to claim 34, wherein the composition contains a dendritic cell activating agent or adjuvant other than the antigen binding protein, fusion protein, or conjugate.

36. A method of modulating an immune response in a subject, the method comprising administering to the subject an antigen binding protein according to any one of claims 1 to 20, a fusion protein according to claims 21, 22, 25 to 30, a conjugate according to any one of claims 23 to 30, nucleic acid of claim 31, vector of claim 32, cell of claim 33, or pharmaceutical composition according to claim 34 or 35, thereby modulating an immune response in a subject.

37. A method according to claim 36, wherein the immune response to an antigen is induced and/or enhanced.

38. A method according to claim 36 or 37, wherein the immune response is modulated by enhancing a helper T cell response.

39. A method according to any one of claims 36 to 38, wherein the immune response is modulated by the activation of CD4+ and/or CD8+ T cells.

40. A method according to any one of claims 36 to 38, wherein the immune response is modulated by enhancing B cell antibody production.

41. A method according to any one of claims 36 to 38, wherein the immune response is modulated by generating a memory response.

42. A method of treating and/or preventing a disease involving dendritic cells or precursors thereof, the method comprising administering to the subject an antigen binding protein according to any one of claims 1 to 20, a fusion protein according to claims 21, 22, 25 to 30, a conjugate according to any one of claims 23 to 30, nucleic acid of claim 31, vector of claim 32, cell of claim 33, or pharmaceutical composition according to claim 34 or 35, thereby treating and/or preventing a disease involving dendritic cells or precursors thereof.

43. A method of claim 42, wherein the disease involving dendritic cells or precursors thereof is selected from the group consisting of cancer, an infection, an autoimmune disease or an allergy, preferably, the infectious disease is any one or more of coronavirus (e.g. SARS-CoV-2), influenza, dengue, hand-foot-mouth disease.

44. Use of an antigen binding protein according to any one of claims 1 to 20, a fusion protein according to claims 21, 22, 25 to 30, a conjugate according to any one of claims 23 to 30, nucleic acid of claim 31, vector of claim 32, cell of claim 33, or pharmaceutical composition according to claim 34 or 35 for the manufacture of a medicament for modulating an immune response in a subject.

45. Use of an antigen binding protein according to any one of claims 1 to 20, a fusion protein according to claims 21, 22, 25 to 30, a conjugate according to any one of claims 23 to 30, nucleic acid of claim 31, vector of claim 32, cell of claim 33, or pharmaceutical composition according to claim 34 or 35 for the manufacture of a medicament for treating and/or preventing a disease involving dendritic cells or precursors thereof in a subject.

46. An antigen binding protein according to any one of claims 1 to 20, a fusion protein according to claims 21, 22, 25 to 30, a conjugate according to any one of claims 23 to 30, nucleic acid of claim 31, vector of claim 32, cell of claim 33, or pharmaceutical composition according to claim 34 or 35 for use in modulating an immune response to an antigen in a subject.

47. An antigen binding protein according to any one of claims 1 to 20, a fusion protein according to claims 21, 22, 25 to 30, a conjugate according to any one of claims 23 to 30, nucleic acid of claim 31, vector of claim 32, cell of claim 33, or pharmaceutical composition according to claim 34 or 35 for use in treating and/or preventing a disease involving dendritic cells or precursors thereof

48. A method of enriching-dendritic cells, or a subset or precursors thereof, from a sample comprising;
(i) contacting a sample comprising dendritic cells or precursors thereof with an antigen binding protein according to any one of claims 1 to 20, a fusion protein according to claims 21, 22, 25 to 30, a conjugate according to any one of claims 23 to 30, nucleic acid of claim 31, vector of claim 32, cell of claim 33, or pharmaceutical composition according to claim 34 or 35, and
(ii) isolating cells bound to the antigen binding protein, fusion protein, conjugate of the invention.

49. A method of detecting dendritic cells, or a subset or precursors thereof, in a sample comprising;
(i) contacting a sample comprising dendritic cells or precursors thereof with an antigen binding protein according to any one of claims 1 to 20, a fusion protein according to claims 21, 22, 25 to 30, a conjugate according to any one of claims 23 to 30, nucleic acid of claim 31, vector of claim 32, cell of claim 30, or pharmaceutical composition according to claim 34 or 35,
(ii) detecting cells bound to the antigen binding protein, fusion protein, conjugate of the invention.

50. A method according to claim 48 or 49, wherein the dendritic cells express one or more of the following markers CLEC9A, HLADR and BDCA3.
